(19) United States
(12) Patent Application Publication
(10) Pub. No.: US 2016/0355951 A1
(43) Pub. Date: Dec. 8, 2016

(54) CORE-SHEATH FIBERS AND METHODS OF MAKING AND USING SAME

(71) Applicant: Arsenal Medical, Inc., Watertown, MA (US)

(72) Inventors: Quynh Pham, Methuen, MA (US); Xuri Ray Yan, Brighton, MA (US); Abby Defeault, Boston, MA (US); Toby Freyman, Waltham, MA (US); Joseph Lomakin, Cambridge, MA (US); Gregory T. Zugates, Chelmsford, MA (US)

(21) Appl. No.: 14/603,485

(22) Filed: Jan. 23, 2015

Related U.S. Application Data
(63) Continuation-in-part of application No. 14/211,742, filed on Mar. 14, 2014.
(60) Provisional application No. 61/852,224, filed on Mar. 15, 2013.
(60) Provisional application No. 61/861,629, filed on Aug. 2, 2013.

(51) Int. Cl. D01F 8/02; D01F 8/16; D01D 5/00; A61L 15/28; A61L 15/22; D01F 8/14; D01F 8/10

(52) U.S. Cl. CPC D01F 8/02; D01F 8/14; D01F 8/16; D01F 8/10; A61L 15/28; A61L 15/225; D01D 5/0038; D10B 2401/022; D10B 2331/30; D10B 2331/041

(57) ABSTRACT

According to one aspect of the invention, multicomponent fiber comprising (a) a polymeric core that comprises a core-forming polymer and (b) a polymeric sheath that comprises a hydrophilic polymer, wherein the core-forming fiber is more hydrophobic than the hydrophilic polymer and wherein the polymeric core, the polymeric sheath, or both, further comprises a hydrophilic excipient material. Other aspects of the present invention pertain to methods of forming such multicomponent fibers. Still other aspects of the present invention pertain to meshes and other articles that are formed using the multicomponent fibers.

### RELATED APPLICATIONS

This application is a continuation-in-part of U.S. application Ser. No. 14/211,742, filed Mar. 14, 2014, entitled “Core-Sheath Fibers and Methods of Making and Using Same” which claims the benefit of U.S. Provisional Application No. 61/852,224, filed Mar. 15, 2013, entitled “Systems and Methods for the Production of Silicone Fibers using Coaxial Electrospinning” and U.S. Provisional Application No. 61/861,629, filed Aug. 2, 2013, entitled “Biocomponent Elastomeric-Hydrogel Fibers,” each of which is incorporated herein by reference in its entirety.

### STATEMENT REGARDING FEDERALLY SPONSORED RESEARCH

This invention was made with government support under Technology Innovation Program Award Number: 70NANB11H004 awarded by the National Institute of Standards and Technology (NIST). The government has certain rights in the invention.

### TECHNICAL FIELD

The present disclosure relates, among other things, to core-sheath fibers, to methods of making core-sheath fibers and to devices and applications associated with core-sheath fibers.

### BACKGROUND

Fibers and collections of fibers have been used as materials in various industrial applications, including applications in medicine and surgery ranging from sutures to wound dressings to skin grafts to arterial grafts, among many others. These applications are based on the unique properties of fibers as materials.

### SUMMARY OF THE INVENTION

According to one aspect of the invention, multicomponent fiber are provided, which comprise (a) a polymeric core that comprises a core-forming polymer and (b) a polymeric sheath at least partially surrounding the polymeric core that comprises a sheath-forming polymer that is different than the core-forming polymer. Examples of core-forming polymers include, for instance, crosslinked polysiloxanes and thermoplastic polymers, among others. Examples of sheath-forming polymers include, for instance, solvent-soluble polymers, degradable polymers and hydrogel-forming polymers, among others.

Other aspects of the present invention pertain to methods of forming such multicomponent fibers. For example, in various preferred embodiments, the multicomponent fibers are formed using coaxial electrospinning techniques.

Still other aspects of the present invention pertain to meshes and other articles that are formed using the multicomponent fibers.

These and many other aspects and embodiments of the present invention will become immediately apparent to those of ordinary skill in the art upon review of the Detailed Description and Claims to follow.

### BRIEF DESCRIPTION OF THE DRAWINGS

FIG. 1 shows a photomicrograph of a cross-section of the PLGA/PDMS sheath/core fibers formed in accordance with an embodiment of the invention.

FIG. 2 shows the PDMS fibers of FIG. 1 after sheath layer removal.

FIGS. 3A-FIG. 3D show top-down and cross-sectional photomicrographs of PLGA/PDMS sheath/core fibers formed in accordance with an embodiment of the invention, both before sheath removal (FIGS. 3A and 3C) and after sheath removal (FIGS. 3B and 3D).

FIG. 4 shows an image of water droplet (left) and an oil droplet (right), placed on a PDMS mesh in accordance with the present invention.

FIG. 5 is a stress-strain diagram illustrating mechanical properties of a PDMS mesh in accordance with the present invention as compared to a cast PDMS film.

FIGS. 6A-6C show cross-sectional photomicrographs of PLGA/PDMS sheath/core fibers that were electrospun at three differing sheath:core flow rates, in accordance with an embodiment of the invention.

FIG. 7A shows photomicrographs of PVP/PDMS sheath/core fibers formed in accordance with an embodiment of the invention, which show: a cross-section of core-sheath fibers where the PVP cured at 100° C.; FIG. 7B the same fibers as in FIG. 7A after they have undergone water extraction; FIG. 7C a cross-section of core-sheath fibers where the PVP cured at 150° C.; FIG. 7D the same fibers as in FIG. 7C after they have undergone water extraction.

FIG. 8 shows FTIR (Fourier transform infrared spectroscopy) scans of a pure PDMS film, a pure PVP film and a PVP/PDMS sheath/core fiber formed in accordance with an embodiment of the invention (cured at 100° C.), when dry and when wet.

FIG. 9 shows FTIR scans of a pure PDMS film, a pure PVP film and a PVP/PDMS sheath/core fiber formed in accordance with an embodiment of the invention (cured at 150° C.), when dry and when wet.

FIG. 10 is a stress-strain diagram illustrating mechanical properties of PVP/PDMS sheath/core fibers formed in accordance with an embodiment of the invention (cured at 100° C. and 150° C.), when dry and when wet.

FIGS. 11A and 11B shows balloon formed from a hydrated PVP-PDMS fiber mesh cured at 100° C., in accordance with an embodiment of the invention, at two levels of expansion.

FIG. 12A-FIG. 12D shows photomicrographs of fibers with a hydrophilic polyurethane (HLPU) sheath and a more hydrophobic polyurethane (HBPU) core, also referred to herein as HLPU/HBPU sheath/core fibers, formed at four HLPU:HBPU ratios, in accordance with an various embodiment of the invention.

FIG. 13 shows swelling and tensile strength as a function of HLPU content for meshes formed from HLPU/HBPU sheath/core fibers formed in accordance with various embodiments of the invention.

FIG. 14 shows swelling and shrinkage as a function of HLPU content for meshes formed from HLPU/HBPU sheath/core fibers formed in accordance with various embodiments of the invention.

FIG. 15 shows swelling for meshes formed from four different HLPU/HBPU sheath/core fibers formed in accordance with the invention (Formulations A-D), as well as two commercially available wound dressings.

FIG. 16 shows wet tensile strength for meshes formed from four different HLPU/HBPU sheath/core fibers formed in accordance with the invention (Formulations A-D), as well as two commercially available wound dressings.

FIG. 17 shows shrinkage for meshes formed from four different HLPU/HBPU sheath/core fibers formed in accordance with the invention (Formulations A-D), as well as two commercially available wound dressings.

FIGS. 18A and 18B show photomicrographs of a mesh formed from HLPU/HBPU sheath/core fibers before and after annealing, respectively, in accordance with an embodiment of the invention.

FIG. 19 shows phosphate buffered saline (PBS) retention for meshes formed from annealed (B Annealed) and non-annealed (B Normal) HLPU/HBPU sheath/core fibers formed in accordance with the invention, as well as two commercially available wound dressings.

FIG. 20 shows shrinkage/expansion for meshes formed from annealed (B Annealed) and non-annealed (B Normal) HLPU/HBPU sheath/core fibers formed in accordance with the invention, as well as two commercially available wound dressings.

FIG. 21 is a photomicrograph of HLPU/HBPU sheath/core fibers with encapsulated silver nanoparticles.

### DETAILED DESCRIPTION OF THE PREFERRED EMBODIMENTS

In accordance with one aspect of the present disclosure, multicomponent fibers are provided which comprise a polymeric core and a polymeric sheath at least partially surrounding (i.e., encapsulating) the core.

As used herein, “fibers,” “microfibers,” and “nanofibers” are used synonymously to refer to elongated structures that differ only by size (with “microfibers” indicating fibers that have cross-sectional diameters on the order of microns to hundreds of microns, “nanofibers” indicating fibers that have cross-sectional diameters on the order of nanometers to hundreds of nanometers, and “fibers” indicating fibers of any size).

Fibers in accordance with the present disclosure can thus be formed in a wide variety of sizes. Preferred overall fiber diameters range from 0.05 to 50 microns (μm) (e.g., ranging from 0.05 to 0.1 to 0.25 to 0.5 to 1 to 2.5 to 5 to 10 to 25 to 50 microns), more preferably 0.1 to 20 microns, among other possible dimensions. Preferred core diameters range from 0.01 to 10 microns (e.g., ranging from 0.01 to 0.025 to 0.05 to 0.1 to 0.25 to 0.5 to 1 to 2.5 to 5 to 10 microns), among other possible dimensions. Preferred sheath thicknesses range from 0.02 to 25 microns (e.g., ranging from 0.02 to 0.05 to 0.1 to 0.25 to 0.5 to 1 to 2.5 to 5 to 10 to 25 microns), more preferably ranging from 0.2 to 18 microns, among other possible dimensions.

The ratio of the sheath volume to core volume can vary widely. Preferred sheath volume:core volume ratios range, for example, from 100:1 to 1:100, among other values, for example ranging from 100:1 to 50:1 to 25:1 to 10:1 to 5:1 to 2:1 to 1:1 to 1:2 to 1:5 to 1:10 to 1:25 to 1:50 to 1:100.

Multicomponent fibers in accordance with the present disclosure can be formed using various fiber spinning techniques, including various melt spinning and solvent spinning methods. Thus, although solvent spinning techniques, and more particularly, electrostatic solvent spinning techniques, are detailed herein, the invention is not limited to such techniques. Further exemplary techniques for forming multicomponent fibers include hot melt spinning, melt electrospinning, centrifugal fiber spinning, wet spinning, dry spinning, gel spinning, gravity spinning, extrusion, extrusion spinning, and rapid prototyping, among others. Using these and other techniques, multicomponent fibers may be formed that comprise (a) a polymeric core that comprises a core-forming polymer and (b) a polymeric sheath at least partially surrounding the polymeric core that comprises a sheath-forming polymer that is different than the core-forming polymer.

Electrospinning is a process that uses an electrical charge to draw very fine, typically micro- or nano-scale, fibers from a liquid. Solvent electrospinning utilizes an electrical force applied to a polymer solution to induce electrospinning jets. As streams associated with the jets travel in the air (or other atmosphere), evaporation of the solvent results in a single long polymer fibers deposited on a grounded collector. The collected fibers can result in the formation of a mesh which may be used in various technologies in medical and non-medical industries including, for example, drug delivery devices, tissue engineering, nano-scale sensors, wound dressings, self-healing coatings, and filters, among many others.

As used herein, a “mesh” is a structure that is formed by a collection of one or more fibers interlaced to form a three dimensional network. Meshes include woven and non-woven meshes.

Meshes in accordance with the present disclosure can vary widely in thickness with preferred thicknesses ranging from 10 to 5000 microns (e.g., ranging from 10 to 25 to 50 to 100 to 250 to 500 to 1000 to 2500 to 5000 microns), among other values.

Meshes in accordance with the present disclosure can vary widely in porosity. In certain embodiments, the meshes of the present disclosure have a porosity of 99% or less, for example, ranging from 99% to 90% to 80% to 70% to 60% to 50% to 40% to 30% to 20% to 10% or less. Porosity can be measured by determining the volume of the polymer and dividing that quantity by the volume of the mesh. In this regard, Polymer volume=Mesh mass÷Polymer density; Mesh volume=Mesh length×Mesh width×Mesh thickness=Mesh area×Mesh thickness; and Mesh porosity=(Mesh volume−Polymer volume)÷Mesh volume. In various embodiments, the porosity of a given mesh may be reduced by annealing the mesh at a temperature and for a time wherein a decrease in mesh porosity is observed.

### Electrospinning

Conventionally, core-sheath electrospinning, also referred to herein as coaxial electrospinning, uses two concentric needles to separately deliver two solutions, specifically, an inner core polymer solution and an outer sheath polymer solution. The core solution is delivered through the inner needle whereas the sheath solution is delivered through the outer needle. Upon activation of an electric field, the two different polymer solutions are ejected in a continuous stream toward a grounded collector; this forms a single core-sheath Taylor cone at the needle tip, leading to the formation of a core-sheath fiber. The creation of core-sheath fibers using needles, however, has limited throughput.

In certain embodiments, core-sheath fibers are generated using a high-throughput core-sheath needleless electrospinning fixture, which utilizes one or more slits on the surface of a hollow vessel to co-localize numerous materials to multiple sites that form Taylor cones, thereby promoting the formation of multiple electrospinning jets and thus multiple electrospun fibers. The slits on the surface of the hollow vessel thus may generate high-throughput production of core-sheath fibers. For further information, see e.g., U.S. Patent Pub. No. 2012/0193836 to Sharma et al. and U.S. Patent Pub. No. 2013/0241115 to Sharma et al., the disclosures of which are hereby incorporated by reference.

In electrospinning, each jet that forms thus leads to one long continuous fiber that gets collected. In a typical operation of the needleless fixture, there are approximately 10 jets that form along the length of the slit; the collected mesh is therefore comprised of approximately 10 very long fibers intertwined with one another. In contrast, during the operation of open bath free surface electrospinning used in the high-throughput core-sheath needleless electrospinning fixture, hundreds of jets form and disappear with each rotation of the drum. Thus, the resulting mesh consists of thousands of relatively short fibers.

The design of the needleless electrospinning fixture takes into account processing parameters that may enable greater control over fiber diameter. For example, in addition to the solution properties, solution flow rates can be manipulated to control fiber diameter. Furthermore, the number of jets produced can also be controlled, which may lead to differences in fiber diameter.

The fibers of any embodiment of the present disclosure may thus be collected in a non-woven mesh form. However, alternate embodiments include fibers that are collected as aligned fibers (as through gap alignment or rotating drum), twisted yarns, ropes, in a pattern, or any other method of fiber collection known in the art of electrospinning.

Fibers with Silicone Components

Various aspects of the invention pertain to multicomponent fibers that are formed using silicone polymers (also referred to herein as “silicones”, “siloxane polymers” or “polysiloxanes”). For example, in certain embodiments, multicomponent fibers are formed that comprise (a) a polymeric core that comprises one or more silicone polymers and (b) a polymeric sheath at least partially encapsulating the core that comprises one or more additional polymers other than silicone, or vice versa.

The present disclosure is applicable to all siloxanes (i.e., compounds with —Si—O—Si linkages), including polysiloxanes, which are formed from multiple siloxane units,

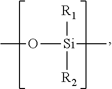

where R₁ and R₂ are organic radicals, for example, linear, branched or cyclic alkyl groups (e.g., methyl groups, ethyl groups, propyl groups, isopropyl groups, butyl groups, isobutyl groups, sec-butyl groups, tert-butyl groups, cyclohexyl groups and so forth), which may be substituted or unsubstituted, as well as substituted or unsubstituted aryl groups (e.g., phenyl groups, p-, m- or o-alkyl-substituted phenyl groups, and so forth). R₁ and R₂ can be the same or different.

In various embodiments, polysiloxanes including as PDMS can be functionalized by a variety of mechanisms (e.g. plasma, UV, CVD, etc.) to modify the surface properties (e.g. hydrophobicity, etc.) or provide specific chemical interactions (e.g. antibody binding). Fibers can be functionalized resulting in immobilized biomolecules on the surface and/or in the bulk. Functionalization can provide many new properties to the material, including biological effects, sensor applications. Microfibers and nanofibers further enhance these benefits by providing high surface areas and small pores, for example.

In this regard, functional groups polymerized as pendant groups attached to the siloxane (e.g., hydrides, hydroxyls, amines, isocyanates, epoxies, etc.) may be used to add chemical activity and diversity and to modify mechanical properties, swelling and solvent resistance, and refractive index, among other properties. The coaxial electrospinning of polysiloxanes as described herein may be combined with functionalization to obtain silicone microfibers and nanofibers with different properties, making them useful in additional applications. For example, treatments which make the fibers more hydrophilic will provide elastic, durable filters which wet more readily. In some embodiments, a functionalizing moiety for the PDMS is incorporated into the fiber. Upon curing, the functional moiety in the fiber becomes incorporated into the PDMS through siloxane chemistry. This allows for one-step functionalizing of the PDMS. In one specific embodiment, PDMS surfaces can be functionalized with biotin groups by adding biotinylated phospholipids to the PDMS prepolymer before curing, as described in Bo Huang et al., “Phospholipid biotinylation of polydimethylsiloxane (PDMS) for protein immobilization,” Lab Chip, 2006, 6, 369-373. These biotin groups can then be further modified with avidin-conjugated to a species of interest, for example, proteins, antibodies or fragments thereof, to functionalize the silicone surface. This may be useful, for example, in removing proteins from a liquid (e.g. protein separation) or in medical implants where preferential binding of certain proteins is advantageous (e.g. improved endothelial cell interactions).

It is noted that some classes of polymers, including various siloxane polymers, are difficult to electrospin due to their low molecular weight and flowability. In this regard, various polysiloxanes remain flowable until they are crosslinked, which does not allow for sufficient polymer chain entanglement for fibers to form.

For example, polydimethylsiloxane (PDMS) is a silicon-based organic polymer belonging to a larger group of siloxane polymers as indicated above, which commonly exhibit properties of elasticity and durability. The ability to manufacture fibers and constructs made from PDMS and other siloxane polymers that exhibit such properties, along with an ability to control the fiber diameter, is highly advantageous in medical technologies as well various other applications. Although attempts have been made to electrospin PDMS fibers, the techniques developed thus far use blended polymer systems (i.e. not pure PDMS) and there are currently no electrospinning methods known to the inventors for manufacturing pure PDMS fiber constructs such as meshes.

Thus, in some aspects of the present disclosure, core-sheath electrospinning techniques are provided, which can be used form fibers that comprise silicone materials that have not been previously electrospun using known techniques. The fibers formed by the techniques described herein comprise a silicone material as the core material, and a different polymer material as the sheath material. After fiber formation and/or collection, the core-sheath fibers are typically crosslinked by a suitable mechanism. For example, the fibers may be cured overnight at room temperature or for a few hours at temperatures up to 100° C., among other crosslinking techniques.

In certain embodiments, the polymeric sheath may be formed from hydrophilic or hydrogel materials, which are discussed in more detail below.

In certain embodiments, the polymeric sheath may be formed from materials that can be dissolved, degraded or otherwise removed from the silicone core, leaving behind pure silicone fibers. Examples of such materials include degradable polymers and solvent-soluble polymers, including water-soluble polymers.

Examples of degradable polymers include one or more of the following, among others: (a) polyester homopolymers and copolymers such as polyglycolide (PGA) (also referred to as polyglycolic acid), polylactide (PLA) (also referred to as polylactic acid) including poly-L-lactide, poly-D-lactide and poly-D,L-lactide, poly(lactide-co-glycolide) (PLGA), polycaprolactone, polyvalerolactone, poly(beta-hydroxybutyrate), polygluconate including poly-D-gluconate, poly-L-gluconate, poly-D,L-gluconate, poly(p-dioxanone), poly(lactide-co-delta-valerolactone), poly(lactide-co-epsilon-caprolactone), poly(lactide-co-beta-malic acid), poly(beta-hydroxybutyrate-co-beta-hydroxyvalerate), among others, (b) polycarbonate homopolymers and copolymers such as poly(trimethylene carbonate), poly(lactide-co-trimethylene carbonate) and poly(glycolide-co-trimethylene carbonate), among others, (c) poly(ortho ester) homopolymers and copolymers such as those synthesized by copolymerization of various diketene acetals and diols, among others, (d) polyanhydride homopolymers and copolymers such as poly(adipic anhydride), poly(suberic anhydride), poly(sebacic anhydride), poly(dodecanedioic anhydride), poly(maleic anhydride) and poly[1,3-bis(p-carboxyphenoxy)methane anhydride], among others, (e) polyphosphazenes such as aminated and alkoxy substituted polyphosphazenes, among others and (f) amino-acid-based polymers.

Examples of water-soluble polymers include non-crosslinked hydrophilic polymers, which may be selected from homopolymers and copolymers formed from one or more of the following monomers, among others: ethylene oxide, vinyl pyrrolidone, vinyl alcohol, vinyl acetate, vinyl pyridine, methyl vinyl ether, acrylic acid and salts thereof, methacrylic acid and salts thereof, hydroxyethyl methacrylate, acrylamide, N,N-dimethyl acrylamide, N-hydroxymethyl acrylamide, alkyl oxazolines, saccharide monomers (e.g., polysaccharides such as dextran, alginate, etc.), and amino acids (e.g., hydrophilic polypeptides and proteins such as gelatin, etc.). When crosslinked, the preceding hydrophilic polymers are useful as hydrogels.

For normal nonwoven materials, microarchitecture is highly dependent upon fiber diameter. Accordingly, an advantage of this core-sheath manufacturing process in which the sheath is subsequently removed is the ability to obtain pore sizes, porosities and other microarchitectural features. Using the high-throughput core-sheath needleless electrospinning fixture (see, e.g., U.S. Patent Pub. No. 2012/0193836 and U.S. Patent Pub. No. 2013/0241115 to Sharma et al.), the ratio of sheath-to-core thickness can be varied to provide larger pore sizes with smaller fibers or higher porosities with smaller fibers than can be obtained with other fabrication techniques.

Fibers with Hydrogel Components and Components of Varying Hydrophilicity/Hydrophobicity

Various aspects of the invention pertain to multicomponent fibers that are formed using hydrogels. For example, in certain embodiments, multicomponent fibers are formed that comprise (a) a polymeric core that comprises one or more core-forming polymers and (b) a polymeric sheath that comprises one or more hydrophilic or hydrogel-forming polymers.

Various aspects of the invention pertain to multicomponent fibers that comprise (a) a polymeric sheath that comprises one or more hydrophilic polymers and (b) a polymeric core that comprises one or more polymers that are more hydrophobic than the one or more hydrophilic polymers. Conversely, other aspects of the invention pertain to multicomponent fibers that comprise (a) a polymeric core that comprises one or more hydrophilic polymers and (b) a polymeric sheath that comprises one or more polymers that are more hydrophobic than the one or more hydrophilic polymers.

Polymers for use as core and/or sheath polymers include those that, upon immersion in an aqueous medium (e.g., water, PBS, etc.) at 25° C. for one hour have water absorption values ranging anywhere from 0% to 1000% or more water, calculated as (wet weight−dry weight)/dry weight (×100), for example ranging from 0% to 1% to 2.5% to 5% to 10% to 25% to 50% to 100% to 250% to 500% to 1000% or more. As used herein, a “hydrophilic polymer” is one that has a water absorption value ranging from from 5-1000% or more water. A “more hydrophobic” polymer, also referred to herein as a “less hydrophilic” polymer, is defined as a polymer that absorbs less water than a given polymer to which it is being compared.

In some embodiments, core and sheath polymers are selected such that the ratio of the sheath polymer water absorption value relative to the core polymer water absorption value ranges from 2:1 to 100:1 (for example ranging from 2:1 to 5:1 to 10:1 to 20:1 to 50:1 to 100:1), among other possible values, preferably 5:1 to 20:1 in certain embodiments. By way of example, the water absorption value of the sheath polymer in Example 4 below is 500% whereas the water absorption value of the sheath polymer is 50%, yielding a sheath:core water absorption ratio of 10:1.

Hydrogels comprise a three dimensional crosslinked network of hydrophilic polymers which have the ability to absorb substantial amounts of water. Hydrogels have long been used in in many applications in the medical field, ranging from drug delivery to tissue engineering scaffolds. Despite many potential applications, hydrogels have limited utility in healthcare or other fields due to a lack of structural control and a poor understanding of hydrogel mechanical properties. Others in the field have looked into reinforcing hydrogels with a variety of additives. Still others have aimed to reinforce hydrogels by making a polymeric fiber or polymeric fiber construct (e.g. a mesh) and then submersing it in a hydrogel or hydrogel-forming polymer before cross-linking the polymer. Such methods and structures have been generally ineffective, and there remains a need for hydrogel structures with desired properties.

In certain aspects of the present disclosure, electrospinning is used to form a fiber core that comprises one or more fiber-forming polymers at least partially surrounded by a sheath that comprises one or more hydrogel-forming polymers. The resulting composite fiber may be optionally subjected to a crosslinking step (e.g., by application of energy such as heat, visible light or ultraviolet light, by application of a crosslinking agent, etc.) to crosslink the hydrogel-forming polymers, the core-forming polymers, or both. The result is a composite fiber that has mechanical and hydration properties that differ from either material alone. These composite fibers can be gathered, formed or processed into various shapes (e.g., tube, mesh, yarns, etc.) for use as medical devices or other products.

Polyurethanes may be employed as core and/or sheath polymers in various embodiments. Polyurethanes are generally formed from diisocyanates and long-chain diols and, typically, chain extenders. Aromatic diisocyanates may be selected from suitable members of the following, among others: methylenediphenyl diisocyanate (MDI), toluene diisocyanate (TDI), naphthalene diisocyanate (NDI), para-phenylene diisocyanate (PPDI), 3,3′-tolidene-4,4′-diisocyanate and 3,3′-dimethyl-diphenylmethane-4,4′-diisocyanate. Non-aromatic (aliphatic) diisocyanates may be selected from suitable members of the following, among others: hexamethylene diisocyanate (HDI), dicyclohexylmethane diisocyanate (H₁₂MDI), isophorone diisocyanate (IPDI), cyclohexane diisocyanate (CHDI), 2,2,4-trimethyl-1,6-hexamethylene diisocyanate (TMDI), and meta-tetramethylxylyene diisocyanate (TMXDI), among others. Long chain diols include polyether diols (e.g., polyethylene glycol, polyoxypropylene glycol, polytetramethylene ether glycol, etc.), polyester diols (e.g., polybutane diol adipate, polyethylene adipate, polycaprolactone diol, etc.), and polycarbonate diols. Other long-chain diols include diol versions for the hydrophilic polymers listed above. Chain extenders include short chain diols such as 1,4 butane diol, among others.

Polyurethanes other than those described in the prior paragraph, may also be employed as core and/or sheath polymers in various embodiments

Hydrogels for use in the present disclosure include those formed from hydrophilic polymers which are crosslinked via a suitable mechanism, for example, covalently crosslinked and/or non-covalently crosslinked (e.g., by ionic crosslinking, physical crosslinking, etc.).

Examples of hydrophilic polymers which may be crosslinked include various hydrophilic polymers such as those set forth above. Further examples of hydrophilic polymers include hydrophilic polyurethanes (e.g., polyurethanes having hydrophilic segments), which may be physically crosslinked (e.g., via hard segments present in the polyurethanes). Specific hydrophilic polyurethanes include aliphatic, polyether-based polyurethanes and aromatic, polyether-based polyurethanes, among others. It is further noted that the hydrophilic polymers set forth above may be employed as hydrophilic segments in polyurethanes in certain embodiments.

Examples of core-forming polymers, which include thermoplastic polymers and polymers of varying hydrophilicity/hydrophobicity in many embodiments, include silicones (polysiloxanes) such as those described above, thermoplastic polyurethanes such as aliphatic, polyether-based polyurethanes and aromatic, polyether-based polyurethanes, among others, and polyamides (e.g., nylon-6,6, nylon-6, nylon-6,9, nylon-6,10, nylon-6,12, nylon-11, nylon-12, nylon-4,6, etc.), among others. Examples of core-forming polymers further include homopolymers and copolymers (including block copolymers) comprising one or more of the following monomers, among others: (a) unsaturated hydrocarbon monomers (e.g., ethylene, propylene, isobutylene, 1-butene, 4-methyl pentene, 1-octene and other alpha-olefins, isoprene, butadiene, etc.); (b) halogenated unsaturated hydrocarbon monomers (e.g., tetrafluoroethylene, vinylidene chloride, vinylidene fluoride, chlorobutadiene, vinyl chloride, vinyl fluoride, etc.); (c) vinyl aromatic monomers including unsubstituted vinyl aromatic monomers (e.g., styrene, 2-vinyl naphthalene, etc.) and vinyl substituted aromatic monomers (e.g., alpha-methyl styrene), ring-substituted vinyl aromatic monomers; and (d) relatively hydrophobic (meth)acrylic monomers, including alkyl (meth)acrylates (e.g., isopropyl acrylate, butyl acrylate, sec-butyl acrylate, isobutyl acrylate, cyclohexyl acrylate, tert-butyl acrylate, hexyl acrylate, 2-ethylhexyl acrylate, dodecyl acrylate, hexadecyl acrylate, and isobornyl acrylate, isopropyl methacrylate, isobutyl methacrylate, t-butyl methacrylate, cyclohexyl methacrylate, 2-ethylhexyl methacrylate, octyl methacrylate, dodecyl methacrylate, hexadecyl methacrylate, octadecyl methacrylate, isobornyl methacrylate, etc.), arylalkyl (meth)acrylates (e.g., benzyl acrylate, benzyl methacrylate, etc.), and halo-alkyl (meth)acrylates (e.g., 2,2,2-trifluoroethyl acrylate). It is noted that many of the preceding polymers can be employed as segments in polyurethanes in some embodiments.

Advantages associated with providing multi-component fibers with a hydrogel sheath and a core material that differs from the sheath material is that fibers, meshes and other constructions can be formed which have good water absorption and retention properties (as a result of the hydrogel material) coupled with desirable mechanical properties such as strength, elasticity, durability and shrinkage (as a result of the core material).

Fibers with Silicone Core and Removable Sheath

As previously noted, certain aspects of the present disclosure pertain to multicomponent fibers that comprise (a) a polymeric core that comprises one or more silicone polymers and (b) a polymeric sheath that comprises one or more additional polymers other than silicone. In certain embodiments, the polymeric sheath may be formed from materials that can be dissolved, degraded or otherwise removed from the silicone core, leaving behind pure silicone fibers. Examples of such materials include degradable polymers and solvent-soluble polymers (including water soluble polymers) such as those set forth above, among others. As elsewhere herein, the fibers can be formed or processed into various shapes (e.g., tube, mesh, yarns) for use as medical devices or other products.

In some embodiments, a silicon core-forming polymer is co-electrospun with a removable (e.g., dissolvable or degradable) sheath-forming polymer to create novel composite fibers. The electrospinning may achieved by needleless electrospinning, coaxial electrospinning, slit-surface electrospinning, or any other suitable technique known in the art of fiber spinning.

In one preferred embodiment, detailed in Examples 1 and 2 below, fibers are formed with a PDMS core and a biodegradable polymer sheath. Cross-linking of PDMS is performed using a two-part system by mixing the pre-polymer and a cross-linking agent which initiates the cross-linking reaction (exposure to heat accelerates this reaction). As used herein, a “pre-polymer”is a polymer material that is subjected to a cross-linking or other curing process to create a crosslinked polymer. In other embodiments, two-part PDMS systems can be cured by exposure to UV-light. In still other embodiments, two-part PDMS systems can be crosslinked into elastomers through free radical, condensation, or addition reactions. Alternatively, one-part PDMS systems may be used which cure upon exposure to moisture in the atmosphere or photo-curing, among other techniques. Any of these variations in PDMS chemistries, or other polymers that require physical or chemical cross-linking to become a solid, may be used in the fibers and methods described herein.

Thus, although a polysiloxane (i.e., PDMS) is exemplified as a preferred embodiment, other embodiments may use polymers (e.g., thermosetting polymers, etc.) that require cross-linking to become solid. Examples include other polysiloxanes and certain types of polyesters, polyurethanes, polyimides, epoxies, etc.

Although degradable polymers (i.e., poly(lactide-co-glycolides)) are exemplified as preferred embodiments, other embodiments may use other degradable polymers or a solvent-soluble polymer sheath (e.g., formed from a water-soluble sheath material such as uncrosslinked PEO, PVA, gelatin, dextran, carbohydrates, etc.), which may be subsequently removed by dissolution. Embodiments employing aqueous solvents as dissolution agents generally do not result in swelling of PDMS fibers.

In some embodiments, the sheath is etched away using an acid.

Depending on the mechanical properties of the sheath polymer, mechanical disruption may be used to break apart the sheath. Any combination of the described methods, or other suitable means, may be employed to remove the sheath from the underlying core.

In some embodiments, therapeutic agents such as small molecule drugs, anesthetics, procoagulants, anticoagulants, antimicrobials, biologics, RNAi, genetic material, genetic vectors, vaccines, or particles such as silver nanoparticles are within the polysiloxane core.

In some embodiments, a porogen (e.g., selected from salts, sugars, etc.) is incorporated within the polysiloxane core. Upon subsequent sheath removal, the porogen is also removed. This will leave behind a fiber with porosity or rough surface features that may improve hydrophobicity, among other properties. Alternatively, a porogen may be incorporated into the sheath such that after fiber formation, a certain percentage of the porogen is located at the interface of the core and sheath. Upon sheath removal, there is a negative imprint of the porogen on the polysiloxane fiber surface. The surface of polysiloxane fibers can also be roughened by a suitable etching process (e.g., laser etching) or mechanical means.

Additionally, porosity can be introduced to the fibers of the present disclosure as a product of the cross-linking reaction that forms the fiber. For example, isocyanate functionalized PDMS can react with water to form porous foam fibers. Another example is acetoxy functionalized PDMS formulated with sodium bicarbonate. The acetic acid byproduct of the cross-linking reaction can react with sodium bicarbonate can generate gas and porosity, thus allowing for the formation of porous foam fibers.

Manipulation of fiber size can yield different fiber properties. For example, in filtration applications, smaller fibers with larger pores or higher porosity can increase the permeability and surface area. Polysiloxane materials (e.g., PDMS) as described herein provide high durability, thermal and oxidative stability and flexibility in combination with small pore size and high permeability. Additionally, polysiloxane fiber meshes formed in accordance with the present disclosure have high surface area due to the small size fibers, which can promote adhesion and wetting where desired. In some embodiments, these same properties may be useful in medical applications where cell infiltration into fibers is desired. In particular, smaller fiber diameters generally facilitate cellular interaction, ingrowth and proliferation while larger pores and higher permeability generally facilitate nutrient, cytokine and gas exchange while also improving cell migration.

In additional embodiments, the sheath is left on the core in order to form a composite fiber that contains a PDMS core and polymer sheath (e.g., nylon, polyethylene, polystyrene, polycarbonates, etc.) that possesses unique properties. In some embodiments, upon immersion in water, the outer sheath may form a hydrogel to fill the porosity of a PDMS fiber mesh.

In further embodiments, core and sheath polymers are reversed, and a polysiloxane is used as the sheath polymer that coats a core polymer. This allows the formation of bi-component fibers with the polysiloxane on the outside. Additionally, removal of the core polymer results in polysiloxane hollow fibers.

Small diameter fiber meshes can provide higher surface area, higher permeabilities and lower pore sizes than meshes made from larger diameter fibers. The present disclosure thus provides materials which combine the benefits of polysiloxanes such as PDMS and small-diameter fiber meshes. For example, solvent-resistant filters or elastomeric, biocompatible microfiber or nanofiber medical device components (e.g., heart valve leaflets, vascular grafts, stent graft coverings) may be formed.

In this regard, in some embodiments, silicone meshes may be used in heart valve leaflets. Replacement heart valves, in some cases, use synthetic materials to recreate the native leaflets. Native leaflets are thin, highly flexible and durable. In addition to these properties the leaflets need to be nonthrombogenic. Encouraging endothelialization is one of the best ways to provide nonthrombogenic implants. The microfiber architecture provided by electrospun silicone is thought to encourage endothelial cell growth. However, this same porosity may lead to blood passing through the pores of the mesh and reduced blood flow control by the valve. This phenomenon is expected to be temporary, however, as proteins and cells become trapped in the pores. In a preferred embodiment, microfiber meshes of silicone are electrospun to a thickness of between 100 to 1000 microns (um). Target fiber diameters are between 500 nm to 10 um. These meshes are then cut into appropriate shapes and attached to a main body which will be implanted via open or minimally-invasive surgery. Alternate embodiments include: providing a membrane (e.g., silicone, PLGA) either on one side of the mesh or sandwiched between two meshes to prevent blood flow through the mesh; functionalizing the silicone with proteins or antibodies (e.g., CD34, VEGF) to encourage tissue ingrowth and reendothelialization; electrospinning onto a frame (e.g., polymer fiber, metal wire, contoured conductive mesh) to help shape the leaflet and/or provide an attachment to the main body; electrospinning onto a biocompatible fiber structure which will create a composite implant (e.g., fibers provide additional mechanical strength or varying stiffness across the leaflet); and coating or functionalizing the fibers to decrease thrombogenicity (e.g., heparin).

In some embodiments, silicone meshes may be used in stent graft coverings in a method similar to the heart valve leaflet, except that the silicone fibers are electrospun onto a tubular collector to form a tube of silicone microfibers or nanofibers. Preferred mesh thickness is between 100 and 1000 microns. Target fiber diameters are between 500 nm to 10 um. This tube can then be attached to a stent to form the stent graft. Alternatively, the fibers may be electrospun directly onto the stent. Alternate embodiments described for the heart valve are applicable here as well. Advantages include the fact that silicone microfibers and or nanofibers will encourage cellular ingrowth while providing an elastic, biocompatible, durable implant. In some embodiments, silicone meshes may be used in vascular grafts similar to the stent graft design except that the tube is not attached to a stent and the preferred mesh thickness range is larger (100 to 5000 microns).

In some embodiments, silicone meshes may be used in bioengineered blood vessels. Much like the vascular graft above, the silicone mesh may be fashioned into a tube and seeded with cells ex vivo. These cells, typically fibroblasts, smooth muscle cells and endothelial cells, are incubated under various conditions (e.g., pulsatile flow, steady flow, no flow) in nutrient-rich environments to grow tissue on the graft material. The silicone microfibers and nanofibers provide advantages in encouraging cell infiltration and growth as well as provide an elastic character typical of blood vessels. The silicone tube may be used alone or in combination with other natural (e.g., collagen) or synthetic (e.g., PTFE, ePTFE, polyurethane) materials. In other embodiments, the graft is seeded with cells and implanted without significant incubation or implanted without cell seeding. In the latter case, cells from the host will infiltrate and populate the graft.

In some embodiments, silicone meshes may be used in arteriovenous (AV) grafts and shunts. These grafts are used in hemodialysis patients to provide better needle access for repeated dialysis. Silicone microfiber or nanofiber meshes will provide a robust set of mechanical properties as well as encourage cellular ingrowth. The elasticity, durability, biocompatibility and low thrombogenicity of silicone will improve the performance of these grafts. In one embodiment, a silicone microfiber or nanofiber mesh is fashioned into a tube and implanted. This tube may be pre-treated by functionalization or coating with other materials (e.g., heparin, collagen, gelatin, growth factors) to improve integration and cell ingrowth. In other embodiments, the silicone mesh may be combined with other natural or synthetic materials as sheets or meshes to form a composite, layered structure. This layered structure may improve the mechanical properties, the ability to contain blood immediately after implantation or long term durability or performance.

In some embodiments, silicone fibers electrospun into a flat mesh configuration of thickness 500 to 5000 microns may be used in hernia meshes. To improve mechanical properties, a composite may be formed with biocompatible polymer fibers by electrospinning directly onto those fibers in the desired configuration. These fibers may also be provided in a configuration that improves suture-ability of the mesh. In alternate embodiments, the mesh may be functionalized, using the various methods described above, to improve tissue ingrowth or integration.

In some embodiments, silicone meshes may be used in dural covering. In neurosurgical procedures where the dura are compromised, it is desirable to provide a covering to re-seal the membrane. A silicone microfiber or nanofiber mesh, optionally combined with a polymer membrane (e.g., silicone, PLGA, collagen) can be used for this purpose.

In other embodiments, silicone meshes may be used in wound dressing. Challenges for wound dressings include adherence to the wound and permeability to air and water (wound exudates). In one embodiment, silicone is electrospun into a mesh between 100 and 5000 microns thick. Preferred fiber diameters are between 500 nm and 10 microns. The electrospun silicone is non-adherent to the wound and provides high permeability and will be used a wound contacting layer in a dressing. In another embodiment, the silicone dressing is supplied separately and medical staff may place additional gauze or other bandages in layers on top of the silicone dressing. In another embodiment, the silicone mesh is combined with a gauze or other backing material as part of the finished product to absorb fluid and protect the wound. In still other embodiments, the silicone can be fabricated with therapeutic agents such as antibiotics, antifungals, topical pain relievers, disinfectants (e.g., iodine) or the like. Another embodiment provides a silicone mesh that has been treated with or manufactured with a hydrogel sheath (e.g., PEG) to provide moisture to the wound bed. The advantage of the silicone mesh in this case is the high porosity can contain the hydrogel material while aiding in removal when the dressing needs to be removed. In still other embodiments, the silicone mesh is fabricated for use with negative pressure wound therapy. In this case, the mesh is sized to be compatible with these devices and is placed on the wound bed as negative pressure is applied. The high permeability and porosity allow exudate removal as well as a non-adherent dressing when it must be removed. For application in negative pressure wound therapy, the silicone fibers may be electrospun onto a collector with a shape and topography similar to the intended treatment site (e.g., face, hand, etc.). In this way, the dressing can improve the therapy by improved conformance to the wounded tissue.

In some embodiments, silicone meshes may be used in hemostatic applications. For hemostatic applications, the device is configured much like the wound dressings, but the silicone microfibers or nanofibers are fabricated or surface modified with a prothrombotic or procoagulant agent (e.g., thrombin, kaolin, chitosan, fibrin, etc.). The silicone provides a non-adherent dressing that can be removed easily. In addition, the high permeability and porosity allows the blood to penetrate and contact a large amount of the surface area with the prothrombotic agent. This open structure also allows for coagulation factor diffusion back into the wound promoting clot formation. This material may also be integrated as a non-adherent layer on other dressings (e.g., Combat Gauze); for this application the fibers may or may not be manufactured with a prothrombotic or procoagulant agent.

In other embodiments, silicone meshes may be used in filtration applications. Silicone meshes may be used as filters or as part of a filter for air, other gases, liquids, slurries or particles. The high solvent resistance and durability provide advantages over other microfiber and nanofiber filters. In particular, the low pore size and high permeability of electrospun, microfiber nanofiber meshes are desirable for filters. In addition, the elastomeric nature provides a way to clean the filter. Simply stretching the material biaxially, circumferentially or otherwise will increase the pore size. Then, backflow of gas or liquid will provide a method to clear the pores of debris or other material. In a similar manner, cake which forms on the intake side of a filter may be easily removed by stretching the silicone mesh allowing the cake to fall off. The silicone microfiber or nanofiber mesh may be used alone (preferred thickness of 100 microns to 1 cm). Alternately, the silicone mesh may be constructed as part of a layered filter using other commonly available filter materials. In this case, the silicone may be electrospun directly onto another material, placed on the other material during assembly or electrospun onto a wire or other fiber mesh with large openings to provide mechanical support.

In some embodiments, silicone meshes may be used in drug delivery. Drugs may be incorporated into the silicone microfibers or nanofibers for delivery to a patient. In one embodiment a silicone mesh is formed with drug in the silicone solution and is placed on the skin for cutaneous or transcutaneous delivery. In another embodiment, the silicone microfibers or nanofibers are formed into a mesh, tube or other structure and implanted to deliver drugs internally. This could include the mouth or other bodily orifices (e.g., delivery of fluoride, bleach or other whitening substances to teeth).

In other embodiments, silicone meshes may be used in barriers to modulate water penetration for controlled drug delivery. A mesh of polysiloxane fibers such as silicone fibers could act as a barrier to modulate drug release. For example, if a drug delivery device has a large burst, a PDMS mesh (which is relatively hydrophobic) can be placed around the device to prevent or slow water contact with the device. Additionally, since silicone is elastic, expansion of the mesh can lead to changes in its porosity and pore size, resulting in an increase of water so as to cause more drug release.

In some embodiments, silicone meshes may be used in pressure-sensitive adhesive bandages. In this embodiment the silicone microfibers or nanofibers are electrospun from a silicone which has adhesive properties. The mesh can then be applied to skin and will adhere well, but will provide water and air permeability to facilitate natural skin function and health. This material can be used in bandages, as part of a wound dressing or for drug delivery patches.

In some embodiments, silicone meshes may be used for oil-water separation as silicone is known to be relatively hydrophobic. With high pore volume fraction, a silicone microfiber or nanofiber mesh will separate oil from water. The silicone may be surface treated, functionalized or doped with additives to make it more oleophilic or hydrophobic. In this application, the silicone mesh may be used as a filter or placed into oil-water mixtures to remove oil or to separate oil from water. This may be extended to other systems containing hydrophilic and hydrophobic materials or phases. Because the mesh is highly elastic, the mesh can be stretched, squeezed, or compressed to clean/remove the oil from the pores for recovery of the oil and/or reuse of the mesh. Additionally, silicone also absorbs organic solvent and can also be used to separate aqueous from organic solvents. The high surface area of microfiber meshes makes it particularly efficient and appealing for these applications.

In some embodiments, silicone meshes may be used in textiles. Silicone microfibers or nanofibers may also be used in textile applications where high elasticity, durability and permeability is desired. In other applications, the hydrophobicity or liquid repellent nature of silicone microfiber or nanofiber meshes (due to architecture) can be used to provide protection from liquids while still allowing air permeability to enable the skin to “breath”.

In various embodiments, the composite fiber can be collected into aligned fiber bundles like a yarn. These yarns will act as strong, elastic fibers that can be used (e.g., sutures) or processed further, including: twisting multiple yarns together into a rope, weaving multiple yarns together into a woven sheet, tube or other shape, braiding multiple yarns together into a stent, scaffold or other tubular structure.

Fibers with Polymeric Core and Hydrophilic or Hydrogel Sheath

Novel materials can be produced by forming various hydrophilic or hydrogel materials around various polymeric core materials, which act as a reinforcing material for the hydrophilic or hydrogel material. The encapsulated polymer material can impart unique material properties (mechanical, chemical, thermal, etc.) to the hydrophilic or hydrogel material that would otherwise not be possible.

More particularly, in some embodiments, a core-forming polymer is co-electrospun with a hydrophilic or hydrogel-forming polymer to create novel composite fibers with a polymeric fiber core that is at least partially surrounded by a hydrophilic or hydrogel sheath. The electrospinning may achieved by needleless electrospinning, coaxial electrospinning, slit-surface electrospinning, or any other suitable technique known in the spinning art. The result is a composite fiber that has mechanical and hydration properties that are distinct from either material alone. These composite fibers can be gathered, formed or processed into various shapes (e.g., tube, mesh, yarns, etc.) for use as medical devices or other products.

Any appropriate hydrophilic or hydrogel-forming material may be used as the sheath polymer and, like the selection of the polymeric core material, the hydrophilic or hydrogel-forming material can be selected to suit the particular purpose of the composite fiber. For example, with regard to the hydrophilic or hydrogel polymer sheath, crosslinked PVP, PEO, PVA, and hydrophilic polyurethanes, among other polymers, as well as xerogels, aerogels, etc., may be used, among many other possibilities. Other hydrogel polymers include crosslinked versions of hydrophilic polymers such as those listed above.

Similarly, any appropriate polymer may be used for the core-forming polymer, depending on the mechanical or chemical needs at hand. In some embodiments, the fiber core is formed using a relatively hydrophobic polymer. While certain embodiments employ a covalently crosslinked silicon-based organic polymer core (e.g., a polysiloxane such as PDMS), the core polymer does not need to be covalently crosslinked to act as a reinforcing fiber. Thus in other embodiments, thermoplastic polymers such as polyurethanes, PLGA, PCL, nylon, polystyrene, acrylic polymers, polypropylene, polyethylene and fluoropolymers, among others, can be used as the core reinforcing fiber.

Polyurethanes represent a broad class of polymers having a wide range of properties and, as such, can serve as core and/or sheath materials in conjunction with the present disclosure. For example, a thermoplastic polyurethane core may be at least partially enclosed in a hydrophilic or hydrogel polyurethane sheath. Many polyurethane materials exhibit physical cross-linking and thus do not require a separate crosslinking step. Such materials may be used, for example, in conjunction with melt-based or solvent-based spinning processes, among others.

The present inventors have demonstrated this concept in conjunction with polyurethane chemistry by co-electrospinning a hydrophilic polyurethane sheath around a more hydrophobic polyurethane core as detailed in Example 4 below. The resulting composite fiber has mechanical and hydration properties that differ from either material alone.

More particularly, a composite material consisting of a mechanically strong polyurethane core and a hydrophilic polyurethane sheath has been created. The particular technique employed was slit-surface, core-sheath electrospinning. As previously noted, electrospinning creates fibers with small diameters (micro or nanometers) which impart additional benefit and functionality (e.g., softness, high surface area, conformability). However, suitable fibers may also be produced using other techniques including hot melt spinning, melt electrospinning, and centrifugal fiber spinning, among other fiber forming techniques.

As noted above, the pre-polymer of PDMS is difficult to electrospin due to its low molecular weight and flowability, which does not allow for sufficient polymer chain entanglement for fibers to form. In addition, the silicone pre-polymer remains flowable until it is crosslinked, so spinning fibers without some way to preserve the fiber structure is unlikely to result in good fiber formation. The present inventors have overcome this difficulty, particularly for micro and nano-sized fibers, by using coaxial electrospinning to encapsulate PDMS pre-polymer and a cross-linking agent within a polymer sheath. In certain embodiments a hydrogel polymer is used as a polymer sheath material. For instance, in Example 3 below, the core polymer is crosslinked PDMS and the polymer sheath is a crosslinked polyvinylpyrrolidone (PVP).

In some embodiments, the cross-linking of the hydrogel-forming polymer is modified to suit the core materials, as well as the desired properties of the composite fiber. In some embodiments, hydrogel crosslinking is initiated by the application of heat, along with core crosslinking. For example the core polymer may be crosslinked PDMS and the polymer sheath may be a crosslinked polyvinylpyrrolidone (PVP), both of which are crosslinked by the application of heat (see, e.g., Example 3). In other embodiments, methods to initiate cross-linking of the hydrogel polymer (and/or core polymer) could include UV or gamma radiation, freeze/thaw cycles, supercritical drying, and so forth. In still other embodiments, a physically crosslinked hydrogel is selected (see, e.g., Example 4). All of these variations of hydrogel chemistries are within the present disclosure.

A major benefit of this aspect of the present disclosure is that an elastic, durable, biocompatible and mechanically stable construct may be provided for hydrogels so that the many potential benefits of hydrogels can be utilized in applications which require greater mechanical integrity. Another benefit is that methods of forming core-hydrogel fibers are provided, which do not require a separate crosslinking step, due to the physical crosslinking attributes of the polymers selected as the core-forming polymer and/or sheath-forming polymer.

As previously noted, small diameter fiber meshes provide, inter alia, higher surface area, higher permeabilities and lower pore sizes than meshes made from larger diameter fibers. This disclosure thus provides materials which combine the benefits of hydrogels and small-diameter fiber meshes.

As elsewhere wherein, these core-hydrogel fibers can be gathered, formed or processed into various shapes (e.g., tube, mesh) for use as medical devices or other products.

Other materials may also be incorporated into the core or sheath polymer to modify or obtain new properties. For example, water absorbing particles may be included to further improve water retention capabilities or agents which will elute out to provide another benefit.

Thus, in some embodiments, excipient materials are incorporated into the fibers to increase water swelling and retention capacities. In certain embodiments, the excipient-containing fibers may have water absorption values, calculated as (wet weight−dry weight)/dry weight (×100) as noted above, ranging from 100% to 250% to 500% to 1000% to 2500% or more. Excipient materials include hydrophilic excipient materials such as cross-linked and non-cross-linked hydrophilic polymers, including cross-linked and non-cross-linked synthetic hydrophilic polymers such as, for example, PVP, among numerous others. Further cross-linked and non-cross-linked hydrophilic polymers for use as hydrophilic excipient materials include cross-linked and non-cross-linked natural hydrophilic polymers and their derivatives such as, for example, cross-linked and non-cross-linked proteins such as gelatin, among others, and cross-linked and non-cross-linked polysaccharides such as starch, modified starch, cellulose, modified cellulose including carboxymethyl cellulose and salts thereof (e.g., sodium carboxymethyl cellulose, etc.), cross-linked carboxymethyl cellulose (e.g., crosscarmellose) and salts thereof (e.g., crosscarmellose sodium, etc.), among others. These materials can be incorporated as dissolved polymers in the sheath or core during electrospinning. Alternatively, they may be included as particulates that are not soluble or are only partially soluble in the solvents used to produce the fibers. In this case, the excipient materials will present as particles embedded in or projecting from the surface of the finished fibers.

In some embodiments, therapeutic agents such as small molecule drugs, anesthetics, procoagulants, anticoagulants, antimicrobials, biologics, RNAi, genetic material, genetic vectors, vaccines, or particles such as silver nanoparticles are incorporated into the fibers which are released upon hydration.

With regard to applications, in some embodiments, the composite core-hydrogel fiber can be used in heart valve leaflets. Replacement heart valves, in some cases, use synthetic materials to recreate the native leaflets. Native leaflets are thin, highly flexible and durable. In addition to these properties the leaflets need to be non-thrombogenic. Encouraging endothelialization is one of the best ways to provide non-thrombogenic implants. The hydrogel layer sheath along with the microfiber or nanofiber architecture will encourage endothelial cell growth. Upon hydration, the hydrogel layer will swell and fill the pores between the core fibers—thus preventing blood from passing through the pores of the valve. In a preferred embodiment, microfiber meshes of core-hydrogel fibers are electrospun to a thickness of between 100 to 1000 microns. Target fiber diameters are between 500 nm to 10 um. These meshes are then cut into appropriate shapes and attached to a main body which will be implanted via open or minimally-invasive surgery. Alternate embodiments include: functionalizing the core polymer with proteins or antibodies (e.g. CD34, VEGF) to encourage tissue ingrowth and reendothelialization (particularly where a degradable hydrogel is selected); electrospinning onto a frame (e.g. polymer fiber, metal wire, contoured conductive mesh) to help shape the leaflet and/or provide an attachment to the main body; electrospinning onto a biocompatible fiber structure which will create a composite implant (e.g. fibers provide additional mechanical strength or varying stiffness across the leaflet); and coating or functionalizing the fibers to decrease thrombogenicity (e.g. heparin).

In some embodiments, the composite core-hydrogel fiber can be used in stent graft coverings. For example, hydrogel fibers can be used as coverings on stents that are used in left atrial appendage closures. These embodiments are similar to the heart valve leaflet, but the core-hydrogel fibers are electrospun onto a tubular collector to form a tube of microfibers or nanofibers. Preferred mesh thickness is between 100 and 1000 microns. Target fiber diameters are between 500 nm to 10 um. This tube can then be attached to a stent to form the stent graft. Alternatively, the fibers may be electrospun directly onto the stent. Alternate embodiments described for the heart valve concept are applicable here as well. Advantages are that composite core-hydrogel fibers will encourage cellular ingrowth while providing an elastic, biocompatible, durable implant.

In some embodiments, the composite core-hydrogel fiber can be used in vascular grafts. These embodiments are similar to the stent graft design but the tube is not attached to a stent and the preferred mesh thickness range is larger (100 to 5000 microns). Alternatively, these tubular meshes act as a reinforcing cuff for vessels (e.g., vascular autografts for bypass surgeries) or other tubular structures where the mechanical properties of the native tissue have deteriorated, such as in abdominal aortic aneurysms.

In some embodiments, the composite core-hydrogel fiber can be used in bioengineered blood vessels. These embodiments are similar to the vascular graft above, and the core-hydrogel microfiber or nanofiber mesh can be fashioned into a tube and seeded with cells ex vivo. These cells, typically fibroblasts, smooth muscle cells and endothelial cells, are incubated under various conditions (e.g. pulsatile flow, steady flow, no flow) in nutrient-rich environments to grow tissue on the graft material. The core-hydrogel microfibers or nanofibers may provide advantages in encouraging cell infiltration and growth as well as provide an elastic character typical of blood vessels. The core-hydrogel tube may be used alone or in combination with other natural (e.g. collagen) or synthetic (e.g. PTFE, ePTFE, polyurethane) materials. In other embodiments, the graft is seeded with cells and implanted without significant incubation or implanted without cell seeding. In the latter case, cells from the host will infiltrate and populate the graft.

In some embodiments, the hydrogel fibers are used in medical device sealing applications. These mechanically robust, hydrogel fibers and resulting meshes, yarns, tubes, etc. are ideally suited for use to seal interfaces between medical devices and the body, other medical devices or other surfaces requiring a seal. For example, they can be used to provide a seal between an implanted heart valve and the native valve annulus to prevent paravalvular leakage. In one embodiment, the hydrogel fibers are electrospun directly onto the outer surface of the valve stent or fashioned into a mesh, yarn or tube and applied to the valve stent as part of the manufacturing process. Upon implantation the hydrogel absorbs water from the blood which leads to swelling, filing of the space between the implant and the valve annulus and thus sealing around the valve to prevent leakage. The advantage compared to other hydrogels is the favorable mechanical properties and durability lead to a safer and more effective product. Other applications include: providing hydrogel microfibers or nanofibers on the vessel contacting side of a stent graft, vascular graft or other medical device to seal between the graft or other medical device and the vessel wall; providing hydrogel microfibers or nanofibers on the outer or inner diameter of a stent graft to seal between two stent graft components which will be assembled together (e.g., EVAR graft main body and iliac limb extension); providing fibers on the outside of a stent graft to be used as a chimney, snorkel, etc. as part of another stent graft placement; providing hydrogel microfibers or nanofibers on the outer surface of a transcutaneous catheter, ostomy bag, or wire lead to seal between the device and the skin and/or underlying muscle, fat or fascia; providing fibers on the outside of a device designed for implantation into the digestive track to prevent food contact with a segment of the digestive system; providing fibers around an endoscopic or laparoscopic instruments or access tubes to provide a temporary seal with the patient's tissues to prevent bleeding, gas leakage or fluid leakage. For those applications where the device is temporary and will be removed the robust mechanical properties and slippery surface of the hydrogel will aid in removal.

In some embodiments, the hydrogel fibers can be manufactured such that they hydrate only when a strain is applied (see, e.g., Example 3 below). Upon hydration, the fibrous construct increases in volume. This property can be applied to create strain-dependent seals around stent grafts and heart valve cuffs. In some cases, when stent grafts and heart valve cuffs are deployed, they do not make complete conformal contact with the vessel wall or annulus, thereby leaving open spaces between the stent graft and vessel, which in turn may lead to leaks, device failure and poor clinical outcomes. The hydrogel fibers can be used as a ring or stent covering such that during delivery, the hydrogel fiber covering does not wet, but upon stent deployment the fiber covering is strained, resulting in wetting and swelling of the fibers that fill empty spaces where the stent does not make conformal contact with surrounding tissues.

In some embodiments, the hydrogel fibers are used in non-medical sealing. For instance, the core-hydrogel fibers will be useful in providing a seal in non-medical applications in aqueous or non-aqueous environments. For example, in aqueous environments, fibers positioned between two surfaces to be sealed will hydrate upon contact with water then the swelling will seal the surfaces and prevent flow through the microstructure. In non-aqueous applications (e.g., oil transport), the mesh will be hydrated upon installation creating a seal from swelling in between two surfaces and also prevent leakage due to immiscibility with the non-aqueous fluid.

In some embodiments, the composite core-hydrogel fiber can be used in arteriovenous grafts or shunts. These grafts are used in hemodialysis patients to provide better needle access for repeated dialysis. A core-hydrogel microfiber or nanofiber mesh will provide a robust set of mechanical properties as well as encourage cellular ingrowth. The elasticity, durability, potential for biocompatibility and low thrombogenicity will improve the performance of these grafts. In one embodiment, a core-hydrogel microfiber or nanofiber mesh is fashioned into a tube and implanted. This tube can be pre-treated by functionalization or coating with other materials (e.g. heparin, collagen, gelatin, growth factors) to improve integration and cell ingrowth. In other embodiments, a core-hydrogel mesh can be combined with other natural or synthetic materials as sheets or meshes to form a composite, layered structure. This layered structure may improve the mechanical properties, the ability to contain blood immediately after implantation or long term durability or performance.

In some embodiments, the composite core-hydrogel fiber can be used in hernia meshes. Core-hydrogel fibers (e.g., silicone or polyurethane core with a hydrogel sheath) can be electrospun into flat mesh configuration of thickness 500 to 5000 microns. To improve mechanical properties, a composite may be formed with biocompatible polymer fibers by electrospinning directly onto those fibers in the desired configuration. These fibers may also be provided in a configuration that improves suture-ability of the mesh. In alternate embodiments, the mesh may be functionalized to improve tissue ingrowth or integration.

In some embodiments, the composite core-hydrogel fibers can be used in dural coverings. In neurosurgical procedures where the dura are compromised, it is desirable to provide a covering to re-seal the membrane. For example, a core-hydrogel microfiber or nanofiber mesh, optionally combined with a polymer membrane (e.g. silicone, PLGA, collagen) can be used for this purpose.

In some embodiments, the composite core-hydrogel fibers can be used in wound dressing. Challenges for wound dressings include adherence to the wound, wound exudate management and permeability to air and water (wound exudates). For example, hydrogel encapsulated polymer (e.g., silicone or polyurethane) may be electrospun into a mesh between 100 and 5000 microns. Preferred fiber diameters are between 500 nm and 10 microns. The advantage of the reinforced hydrogel is that it provides moisture to the wound bed while also forming a protective layer which does not adhere to the wound. In one embodiment, a hydrogel-polymer dressing is supplied separately and medical staff place additional gauze or other bandages in layers on top of the core-hydrogel fiber dressing. In another embodiment, a core-hydrogel mesh is combined with a gauze or other backing material as part of the finished product to aid in the absorption of fluid and protect the wound. In still other embodiments, a core-hydrogel mesh can be fabricated with therapeutic agents such as antibiotics, antifungals, topical pain relievers, disinfectants (e.g. iodine) or the like. In still other embodiments, a hydrogel-polymer mesh is fabricated for use with negative pressure wound therapy. In this case, the mesh is sized to be compatible with these devices and is placed on the wound bed as negative pressure is applied. The high permeability and porosity allow exudates removal as well as a non-adherent dressing when it must be removed. The hydrogel sheath or core polymer may also be useful in controlling release of therapeutic agents to the wound (e.g., antimicrobials, antibiotics, silver ions, growth factors, analgesics, anesthetics, debridement compounds or enzymes, etc.).

In some embodiments, the composite core-hydrogel fiber can be used in hemostat applications. For hemostatic applications, the device is configured much like the wound dressings, but the hydrogel-polymer microfibers or nanofibers are fabricated or surface modified with a prothrombotic agent (e.g. thrombin, kaolin, chitosan, fibrin). The fiber provides a nonadherent dressing that can be removed easily. In addition, the high permeability and porosity allows the blood to penetrate and contact a large amount of the surface area with the prothrombotic agent. This open structure also allows for coagulation factor diffusion back into the wound promoting clot formation. This material may also be integrated as a non-adherent layer on other dressings (e.g. Combat Gauze); for this application the fibers may or may not be manufactured with a prothrombotic agent.

In some embodiments, the composite core-hydrogel fiber can be used in filtration. Composite core-hydrogel fiber meshes can be used as filters or as part of a filter for air, gases, liquids, slurries or particles. In particular, the low pore size and high permeability of electrospun, microfiber or nanofiber meshes are desirable for filters. In addition, where the fibers are elastomeric, the elastomeric nature provides a way to clean the filter. Simply, stretching the material biaxially, circumferentially or otherwise will increase the pore size. Then, backflow of gas or liquid will provide a method to clear the pores of debris or other material. In a similar manner, cake which forms on the intake side of a filter can be easily removed by stretching the fiber mesh allowing the cake to fall off. The core-hydrogel microfiber or nanofiber mesh may be characterized by high strength and hydrophilicity, thus being useful as a filter, barrier or separating membrane to partition oil content in water. The core-hydrogel microfiber or nanofiber mesh can be used alone (preferred thickness of 100 microns to 1 cm). Alternately, the core-hydrogel mesh can be constructed as part of a layered filter using other commonly available filter materials. In this case, the core-hydrogel may be electrospun directly onto another material, placed on the other material during assembly or electrospun onto a wire or other fiber mesh with large openings to provide mechanical support.

In some embodiments, the composite core-hydrogel fiber can be used in drug delivery. The hydrated core-hydrogel composite material may act as a substantially non-porous yet conformal layer. In one embodiment the core-hydrogel material would be inserted into the target delivery area then inflated with gas or other fluid (e.g., a drug containing solution, etc.) to conform to the internal structure of the target area. Direct, conformal contact of the hydrogel with the surface leads to efficient drug delivery. Alternatively, upon reaching a certain expansion limit on inflation, the pores become stretched and open to allow drug solution to be released. Once deflated, the pores seal back up thus inhibiting drug delivery to areas not being targeted during removal of the device. This approach is particularly applicable for therapeutic delivery to cavities and lumens, such as the sinusoidal space.

In various embodiments, drugs may be incorporated into the core-hydrogel microfibers or nanofibers for delivery to a patient. For example, a core-hydrogel fiber mesh may be formed with drug in the core-forming solution, and placed on the skin for cutaneous or transcutaneous delivery. Fiber meshes of the present disclosure are beneficial in that they provide a means of targeted delivery to difficult orifices such as sinus cavities, intestinal wall or ear canals due to the ability to balloon open for conformal delivery. A tubular or other shaped mesh may also be implanted to provide sustained drug delivery. It may be implanted alone or held in place using another medical device, such as a stent.

In some embodiments, the composite core-hydrogel fibers can be collected into aligned fiber bundles like a yarn. These yarns will act as strong, elastic hydrogel fibers that can be used (e.g., sutures) or processed further, including: twisting multiple yarns together into a rope, weaving multiple yarns together into a woven sheet, tube or other shape, braiding multiple yarns together into a stent, scaffold or other tubular structure. These configurations can be developed into novel medical devices such as hydrogel catheters, introducer sheaths, guide wires, vascular grafts, hernia meshes, etc.

In some embodiments, the composite core-hydrogel fiber can be used in textiles. Core-hydrogel microfibers or nanofibers may also be used in textile applications where high elasticity, durability, water absorption and permeability are desired.

In some embodiments, the composite core-hydrogel fiber can be used in tissue engineering applications. Hydrogels allow for free diffusion of oxygen, nutrients, etc., which is desirable for these purposes. This property is further enhanced, because diffusion not only can occur across the hydrogel bulk, but through the porosity created by the fibrous network. Hydrogels are used extensively in tissue engineering applications due to their promising biocompatibility and hydration properties. A major benefit of the present disclosure is that fibrous hydrogels would allow for better cell attachment and integration to form 3D scaffolds. The hydrogel sheath would allow for cell attachment and in-growth, which could eventually degrade away, while the core polymer fibers would provide more permanent mechanical support. A specific example application of this includes hyaline cartilage repair, in which the hydrogel sheath provides a biocompatible scaffold for stem cells to attach and differentiate into chondrocytes while the porosity provides space for chondrocytic secretion of collagen and ECM components.

In some embodiments, the hydrogel fibers are used as a tissue bulking agent in cosmetic or plastic surgery. The elastic and flexible mechanical properties and high hydration of the hydrogel fibers can be tailored to match that of native tissue for a more natural look and feel. The fibrous nature will integrate with the surrounding tissue such that the bulking agent stays in place and will not become displaced. Furthermore, the hydrogel can be made to be nonbioresorbable and therefore maintain its bulking capacity over time.

In some embodiments, the composite core-hydrogel fiber are used as medical electrodes. The swelling properties of hydrogel allow for conformable and intimate contact with tissue that can lower electrical impedance and improve electrode performance. Furthermore, to improve electrical conductance, the core material can be comprised of a conductive polymer or include electrically conductive particles or ions.

The ballooning and hydration capability of the composite core-hydrogel fibers is a unique property that can be used for the ablation of tissues through the use of microwaves. For example, for ablation within a body cavity (e.g., endometrial, left atrial appendage) or to an irregular surface (e.g., liver, esophagus, sinuses) a mesh of composite core-hydrogel fibers can be inflated with a gas (e.g., carbon dioxide) to make conformal contact with the tissue. Application of microwaves from a source within the balloon will heat the water within the hydrogel membrane, which is in intimate and conformal contact with the cavity or tissue surface, to thermally ablate the surrounding tissue.

This same technique may be extended to other ablation approaches, including hydrothermal (e.g., inflate the balloon with hot water or other hot liquid), chemical (e.g., ablative agent in the hydrogel fibers) or cyroablation (e.g., cold source or liquid nitrogen used to chill the balloon).

The composite core-hydrogel fibers of the present disclosure may also be used to embolize a body lumen. The composite structure provides a fiber or coil that can be inserted into a patient using techniques know to those skilled in the art. The hydrogel properties then swell the fibers to completely fill the body lumen or aneurysm cavity. Two key advantages here are 1) combination of fiber strength and high swelling ratio, and 2) ability to form very small fibers or coils and/or flexible implants.

### EXAMPLE 1

### Fibers with PDMS Core and PLGA Sheath

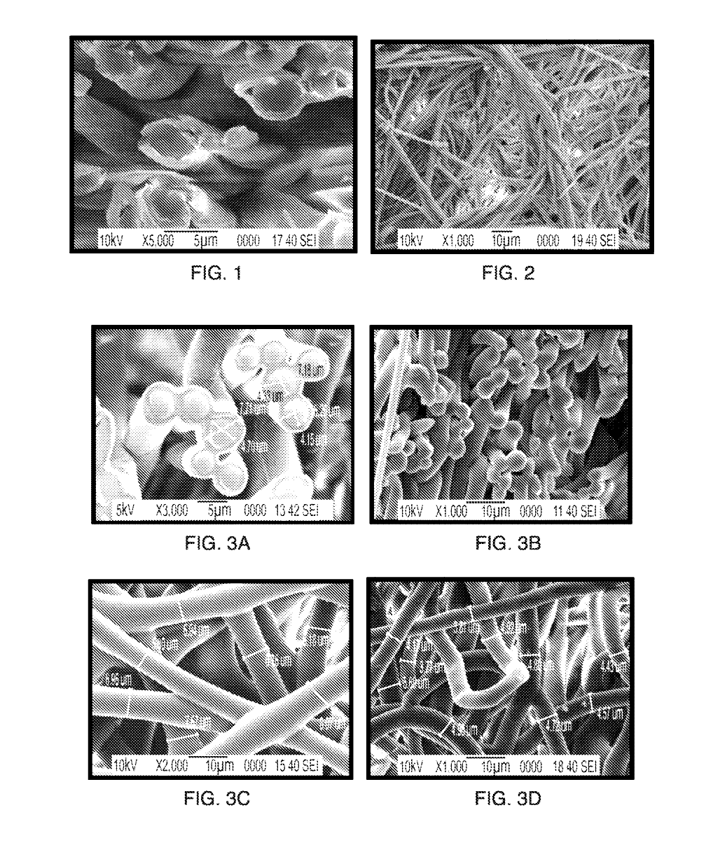

Core/sheath fibers are fabricated in accordance using a high-throughput core-sheath needleless electrospinning fixture. The sheath polymer system was a 3.5 wt % 85/15 poly(L-lactic acid-co-glycolic acid) (PLGA) in 6:1 (by vol) chloroform:methanol solvent. The core polymer consisted of PDMS (Sylgard 184, available from Dow Corning, a two-part liquid system consisting of part A (pre-polymer) and part B (cross-linking agent)) mixed in a 10:1 mass ratio. The sheath solution flow rate was set to 200 ml/h while the core flow rate was set to 20 ml/h. The fibers were deposited onto and collected from a grounded collection plate. The fabricated mesh was then placed in an oven at 100° C. (to accelerate curing) for 3 hours and then immersed in chloroform for 1 hour to dissolve the PLGA sheath. The PDMS fiber mesh swelled to an extent upon exposure to the solvent, but then shrank back to original size after solvent evaporation. FIG. 1 shows an image of the cross-section of the PLGA/PDMS sheath/core fibers after curing. The different polymers in the sheath/core configuration can be observed. FIG. 2 shows the PDMS fibers after sheath layer removal. PDMS fibers were manufactured to be between about 1 and 5 microns in diameter. As described elsewhere herein, however, the diameter of the core PDMS can be tuned by modulating electrospinning parameters.

### EXAMPLE 2

### Further Fibers with PDMS Core and PLGA Sheath

Core-sheath fibers were electrospun with 50/50 poly(D,L-lactic acid-co-glycolic acid) (5050 PDLGA) as the sheath over a PDMS (Sylgard 184) core, as described in Example 1. The sheath solution was an 11 wt % 5050 PDLGA in hexafluoroisopropanol (HFIP). The flow rate for the sheath solution was set at 10 ml/h while the core solution flow rate was set at 1 ml/h. The fibers were subsequently placed in a 60° C. oven for 24 hours to allow the PDMS in the fibers to cure. FIG. 3A shows a core-sheath structure (in cross-section) that was formed. Diameters of the fibers were measured for both top-down and cross-sectional images. The overall fiber diameter of the fibers was approximately 7 microns (see FIGS. 3A and 3C), while the core PDMS diameter was approximately 4.5 micron (see FIGS. 3B and 3D). The 5050 PDLGA sheath was removed under accelerated degradation conditions by immersing the mesh in 12 pH buffer consisting of 1.5% sodium phosphate, 0.1% boric acid, and 0.08% citric acid at 37° C. for 7 days. As can be seen in FIG. 3B, the sheath layer was completely degraded and removed, leaving behind PDMS-only fibers.

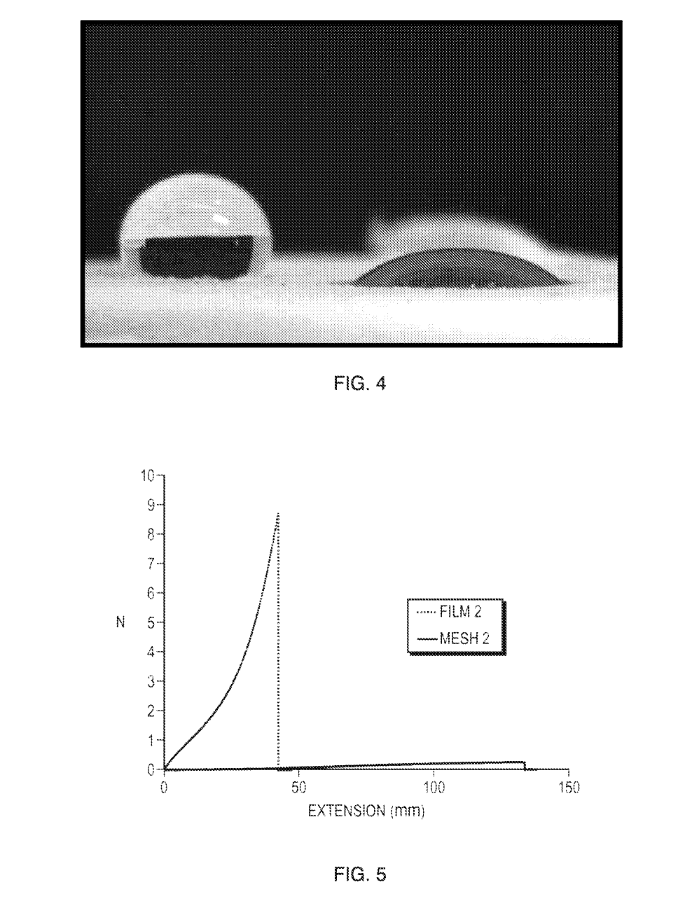

The electrospun fibers and meshes of the present disclosure offer different properties than those formed from traditional methods of constructing PDMS as a cast film. The contact angle of the electrospun PDMS-only mesh was measured to be 110° while a cast film of PDMS had a contact angle of 104°. FIG. 4 shows the hydrophobic and oleophilic nature of the PDMS mesh formed using the electrospinning processes of the present disclosure. A water droplet (left) placed on the mesh remains beaded while an oil droplet (right) wets the mesh and can move throughout the porosity of the mesh. FIG. 5 shows the mechanical properties of the mesh compared to a cast PDMS film. The data indicates that the PDMS fiber mesh exhibits significantly different mechanical properties than a cast film. The modulus of the mesh is significantly lower (0.2 MPa vs 2.0 MPa) while its extension at max loading is significantly higher (300% vs. 122%) relative to the cast PDMS film.

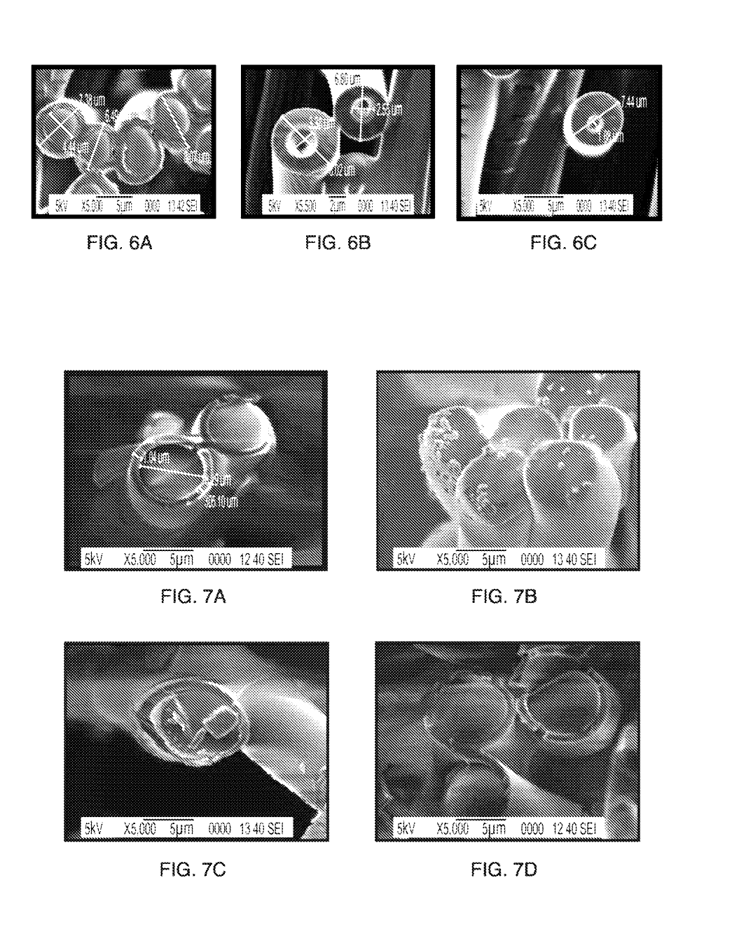

FIGS. 6A-6C show cross-sectional photomicrographs of electrospun fibers of the present disclosure having PDMS in the core and 5050 PDLGA in the sheath. The electrospinning process was carried out at sheath:core flow rates of 10:1, 10:0.25, and 20:0.25 ml/h in order to generate PDMS fibers with different fiber diameters, as shown in the cross-sectional images of FIGS. 6A-6C.

### EXAMPLE 3

### Fibers with PDMS Core and PVP Sheath

Core/sheath fibers were fabricated using a sheath polymer solution of 8 wt % PVP (polyvinyl pyrrolidone) in TFE (trifluoroethanol), while the core polymer solution consisted of Sylgard 184, a two-part liquid system consisting of Part A (pre-polymer) and B (cross-linking agent) mixed in a 10:1 mass ratio. The sheath flow rate was set to 10 mL/h while the core flow rate was set to 2 mL/h.

Meshes were collected on PTFE coated aluminum shims and then cured at either 100° C. or 150° C. for 24 hours. The meshes were then removed from the aluminum shims and submerged in deionized water in which any non-crosslinked PVP was solubilized by the water. The remaining PVP was crosslinked as a robust sheath around the silicone fiber cores, which then formed a hydrogel and swelled to >200% its initial mass, the amount of swelling is proportional to the degree of crosslinking of the PVP (and thus the temperature of the cure). In particular, for the 100° C. sample, swelling (by mass) was measured at 242%±48%, whereas for the 150° C. sample, swelling (by mass) was measured at 401%±76%.

Gel fraction data (% hydrogel) were generated. For the 100° C. sample, the gel fraction was measured at 64%±1%, whereas for the 150° C. sample, the gel fraction was measured at 98%±3%. These data indicate that upon water extraction of non-cross-linked PVP, the 100° C. cured sample loses ˜40% of its mass while the 150° C. sample maintains almost 100% of its mass. This suggests that the PVP sheath is nearly completely cross-linked at 150° C. and may be only partially cross-linked at 100° C.

Similar conclusions can be drawn by cross-sectional analysis with SEM, as illustrated in FIG. 7, which shows: (A) SEM cross-section of core-sheath fibers where the core consists of fully cured PDMS and the sheath is PVP cured at 100° C.; (B) SEM cross-section of the same fibers in (A) except after they have undergone water extraction to remove non-cross linked PVP; (C) SEM cross-section of core-sheath fibers where the core consists of fully cured PDMS and the sheath is PVP cured at 150° C.; (D) SEM cross-section of the same fibers in (C) except after they have undergone water extraction to remove non-cross linked PVP. Before hydration, the two cure temperature samples look identical in core fiber diameter (around 6 um) and in sheath thickness (around 1 um). After hydration and subsequent drying, however, the sheath appears to be almost completely removed in the 100° C. sample while it remains intact in the 150° C. sample.

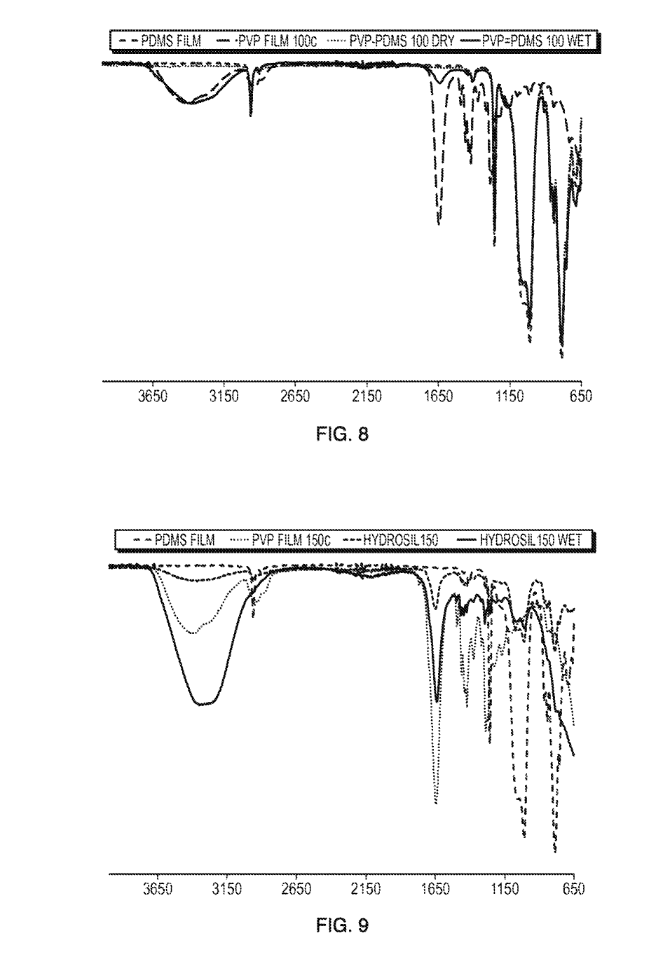

Analysis by FTIR can indicate the presence of PVP in the sample by the existence of an amine peak around 1650 cm⁻¹. Additionally, PDMS does not absorb water so the presence of a broad peak around 3400 cm⁻¹ indicates an O—H bond and therefore the absorption of water by the sample, which would only occur if cross-linked PVP is present. FIGS. 8 and 9 show the spectra obtained from the silicone fiber hydrogels in the wet and dry states as compared to pure PDMS and pure PVP cured at temperatures of 100° C. and 150° C., respectively.

In comparing the spectra for PVP-PDMS cured at 100° C. to pure PDMS and pure PVP it can be confirmed that very little PVP remains in the sample after the initial water extraction. What little PVP that remains can only be detected when the sample is in the hydrated state. The dry PVP-PDMS hydrogel matches nearly perfectly with pure PDMS and absorbs essentially no water from the atmosphere. This supports the mass loss data and observations from the SEM that an essentially undetectable amount of PVP remains on the sample although it still behaves as a hydrogel.

Contrary to the spectra for the PVP-PDMS hydrogels cured at 100° C., the spectra for samples cured at 150° C. show an amine peak and absorbed water even when dry. This is further evidence to support that nearly all of the PVP is cross-linked at this higher temperature and remains in the sample after initial water extraction.

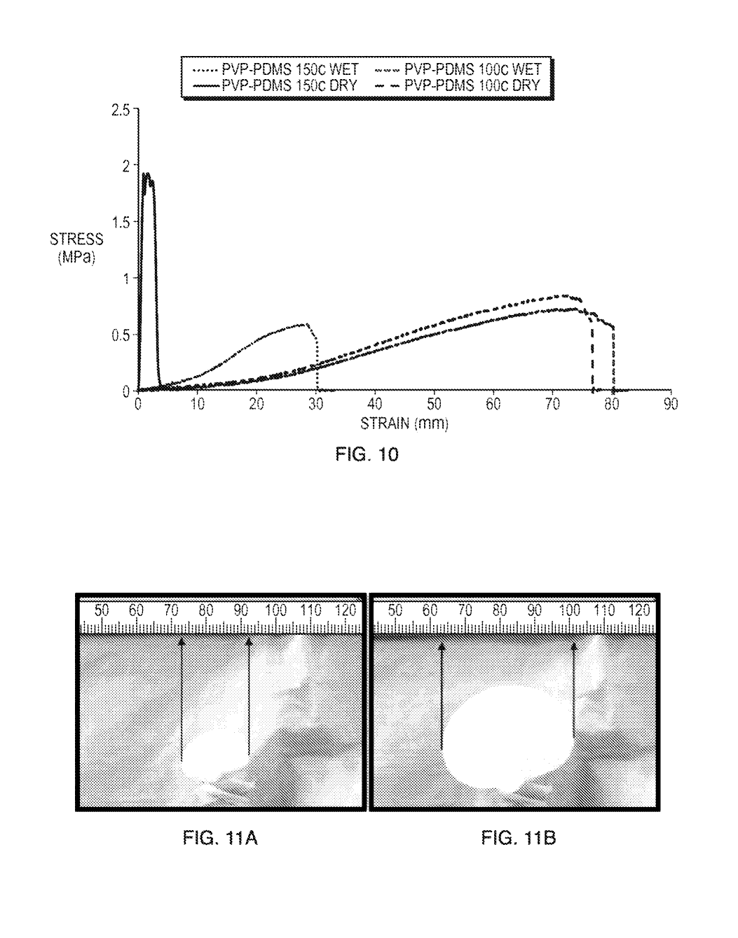

The mechanical properties of the fiber hydrogel are dramatically enhanced due to the presence of a silicone microfiber structure. Tensile properties of hydrogels are rarely reported and difficult to find due to the poor mechanical stability of the same. With silicone fiber reinforcement, the hydrogel has a larger surface area for wetting while also maintaining mechanical integrity and strength. Additionally, the presence of a cross-linked hydrogel layer on the silicone provides another layer of support for the silicone fibers and increases the overall strength of the composite material. FIG. 10 shows the comparison of different formulations of hydrogel-silicone microfiber composites both in the wet and dry states. The samples were cut using a 20 mm die and tested on an Instron® system at a rate of 50 mm/min. It can be seen from this data that temperature of cross-linking can affect the tensile strength and modulus of the material.

This data also supports the previously stated conclusion that very little cross-linked PVP remains on the sample cured at 100° C. given that the mechanical properties appear to be unaffected by the hydration state of the material. Conversely, the PVP-PDMS cured at 150° C. behaves drastically different when it is dry than when hydrated. In the dried state the material has a modulus ˜200 times greater than when it is hydrated (i.e., 75 MPa vs. 0.4 MPa). It also has a much shorter elongation to break (7% vs. 140%). When the material is in the hydrated state the PVP swells and the mechanical properties are driven largely by the PDMS fibers.

Another feature of silicone core/hydrogel sheath fibers is that its mechanical features can change, depending on whether the fibers are wet or dry. For example, a dry mesh will have high air permeability, high porosity and will be opaque. Conversely a hydrated mesh will have lower air permeability (because the swollen hydrogel fills the pores), high water permeability and will be optically clear. Upon reaching its expansion limit of the mesh the pores open up and the gas or liquid flows through the mesh as opposed to bursting it. This ability to expand is also affected by the cure temperature, because as the elongation of the fibers is dependent on cure temperature.

As shown in FIG. 11A, a “balloon” was formed from a mesh of hydrated PVP-PDMS fibers cured at 100° C. If spherical expansion is assumed, then the volume expansion ratio is near 800% when the balloon reaches maximum expansion (see FIG. 11B). At the maximum expansion, the balloon doesn't burst but merely becomes air permeable and allows air to escape through the pores. The air permeability and porosity of the hydrated mesh can be increased upon stretching the mesh to open up the pores. Due to the decreased permeability of the hydrated hydrogel mesh, the material can hold air or water and expand to very high volumes while still maintaining mechanical integrity. In addition, the microfibers provide a flexible balloon that can conform to irregular surfaces, cavities or containers. Compared to a pure PDMS fiber mesh, which expands only about 100% before bursting, the effect of the very low amount of cross-linked PVP on the surface is quite significant. The PVP-PDMS hydrogel cured at 150° C. expands to 450% before becoming air permeable, thus indicating that cure temperature and therefore amount of cross-linked PVP in the sample effects this property.

Due to the unique pairing of a hydrophilic hydrogel sheath with an oleophilic core fiber, the PVP-PDMS fiber mesh swells in both water and oil. Table 1 shows a comparison of the swelling properties of the PVP-PDMS meshes in DI water and Vacuum Pump Oil at different curing temperatures. The 100° C. cured mesh absorbs nearly the same amount of oil as it does water due to the presence of the PDMS fibers and the very small amount of PVP on the surface. The mesh cured at 150° C., on the other hand, absorbs much more water than it does oil, because much more crosslinked PVP is present in this sample.

TABLE 1

Cure Temperature

| | 100° C. | 150° C. |
|---|---|---|
| Water Swelling (%) | 242% | 401% |
| Oil Swelling (%) | 215% | 150% |

In addition to the swelling, ballooning and mechanical strength differences between samples cured at 100° C. and those cured at 150° C., these samples also show a distinct difference in their wettability after the initial hydration (extraction) and drying. The PVP-PDMS fibers cured at 150° C. quickly absorb water and become fully hydrated without any additional handling or manipulation. Meshes cured at 100° C., on the other hand, are more hydrophobic in their dry, unstretched state. In order to hydrate the meshes, they are manipulated (e.g., stretched). In this regard, when water is initially applied to the dry mesh, it beads on the surface. However, as the sample is stretched and manipulated, it eventually becomes fully hydrated.

### EXAMPLE 4

### Fibers with a Polyurethane Core and a Hydrophilic Polyurethane Sheath

In this example, slit-surface, core-sheath electrospinning was employed, in which a hydrophilic aliphatic polyether-based thermoplastic polyurethane (HLPU) was used as the sheath material, while a mechanically stronger more hydrophobic aliphatic polyether-based thermoplastic polyurethane material (HBPU) was used as the core material. The electrospinning solutions were as follows: 4 wt % HLPU in TFE and 6 wt % HBPU in HFIP. Electrospinning was carried out at different sheath:core flow rate ratios. At the flow rate ratios selected, the resulting fiber was composed of HLPU and HBPU in the following HLPU:HBPU weight ratios: (A) 93:7, (B) 82:18, (C) 60:40, and (D) 38:62, respectively.

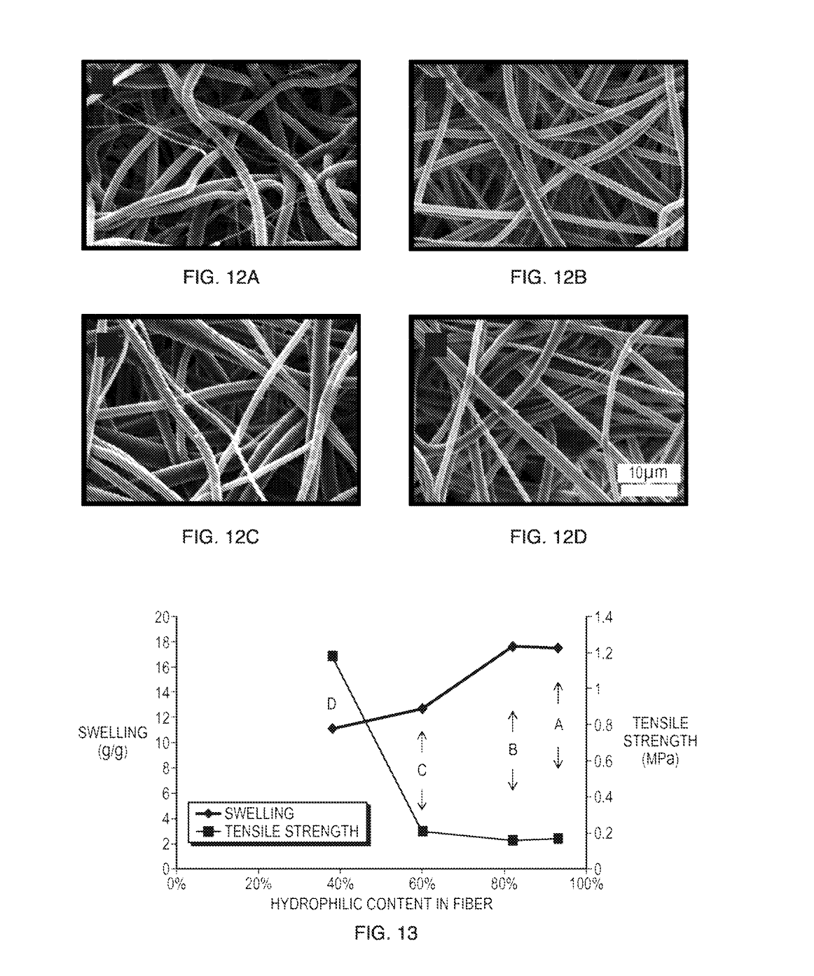

FIG. 12 shows the SEM of the fibers for each composition; fiber diameters for all formulations were approximately 2 microns

Characterization of the meshes included dimensional, and hydration measurements, which are summarized in Tables 2 and 3 below. Mechanical characterization was determined by cutting the meshes into dog-bone shapes and performing tensile testing using an Instron® at a pull rate of 50 mm per minute. Swelling was characterized by immersing samples in phosphate buffered saline (PBS) for at least 20 minutes and the PBS was allowed to drip off before weighing. Swelling was calculated as the (wet weight−dry weight)/dry weight. PBS retention was determined by placing the hydrated material on filter paper and applying a weight equal to 40 mmHg for 30 seconds. The sample was then re-weighed to determine the amount of water lost during testing. The wet tensile strength of the different polyurethane samples are shown in Table 2 and demonstrates an increase in mechanical properties as the amount of HBPU in the fiber is increased. Therefore, by varying the core to sheath material composition, one can modulate the tensile strength.

TABLE 2

| | Formulation | Formulation | Formulation | Formulation |
|---|---|---|---|---|
| | A | B | C | D |
| HLPU:HBPU | 93:7 | 82:18 | 60:40 | 38:62 |
| Ratio | | | | |
| Wet tensile | 0.20 ± 0.03 | 0.15 ± 0.02 | 0.26 ± 0.05 | 1.21 ± 0.08 |
| strength (MPa) | | | | |

Table 3 shows the hydration properties of the different formulations and indicates that sample shrinkage upon hydration and swelling were most impacted by the chemical composition of the fibers. However, PBS retention did not appear to be significantly impacted.

TABLE 3

| | Formulation A | Formulation B | Formulation C | Formulation D |
|---|---|---|---|---|
| HPLU:HBPU Ratio | 93:7 | 82:18 | 60:40 | 38:62 |
| Basis weight (GSM) | 55 ± 4.5 | 92 ± 0.5 | 110.8 ± 16.1 | 94 ± 5.7 |
| PBS absorption (%) | 1750 ± 23 | 1760 ± 57 | 1270 ± 201 | 1110 ± 101 |
| PBS retention (%) | 52 ± 2 | 58 ± 1 | 56 ± 2 | 56 ± 1 |
| Shrinkage (%) | 57 ± 0.5 | 35 ± 2 | 14 ± 4 | 2 ± 6 |

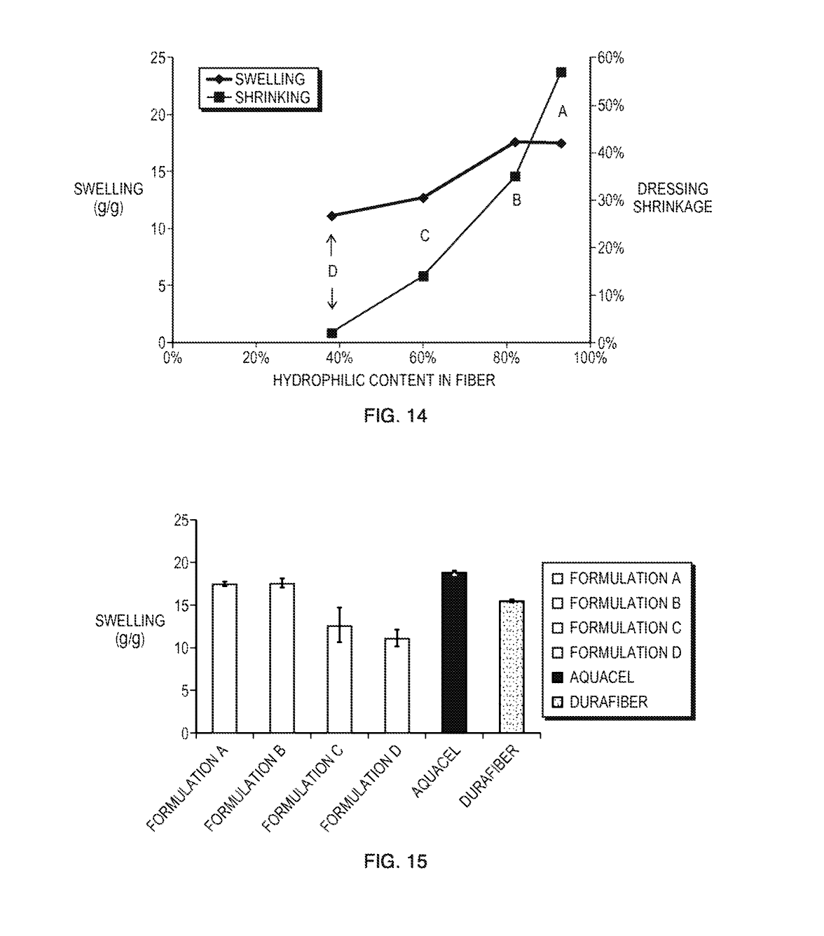

A comparison of the mechanical and hydration properties as a function of HLPU content is shown in FIGS. 13 and 14. FIG. 13 shows that tensile strength increases as the amount of HLPU decreases (and hence the amount of HBPU increases). However, as the tensile strength increases, the amount of PBS absorption decreases as a result of less hydrophilic material being present.

A comparison of the swelling (or PBS absorption) and shrinkage data as a function of the HLPU content further reinforces the utility of using a core-sheath fiber structure to modulate the mechanical and hydration properties. As shown in FIG. 14, there is an increase in swelling capacity as the HLPU content increases; however, dimensional shrinkage (i.e., shrinkage in area) of the mesh is also observed to increase as the HLPU content increases. These data illustrates the formulation space for these materials and shows a correlation between performance of the hydrogel mesh and its chemical composition.

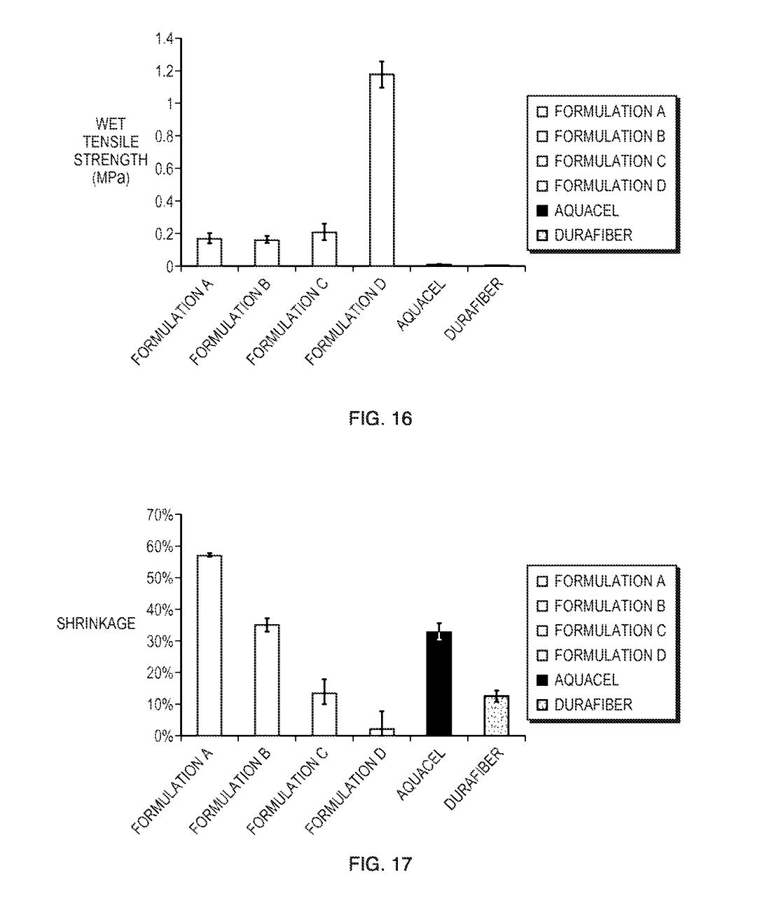

The performance across tensile strength, shrinkage, and swelling has been optimized by varying the sheath to core ratio of the polymeric materials. This is highly advantageous for numerous applications, especially medical applications. For example, hydrogel wound dressings are cut to fit the wound size when dry. These dressings improve wound healing by providing a moist environment and absorb excess wound exudate to prevent leakage. However, excessive shrinkage may result in a dressing which inadequately covers the wound after it starts to absorb liquid. As shown in FIGS. 15, 16 and 17, in comparison with commercially-available wound dressings such as Aquacel® (ConvaTec Inc.) or Durafiber® (Smith & Nephew), a material has been developed which provides equivalent water absorption (see FIG. 15, Formulation A and B), much stronger mechanical properties (see FIG. 16, all Formulations) and has minimal shrinkage (see FIG. 17, Formulations D) or shrinkage that is comparable to those existing products (see FIG. 17, Formulations B and C).

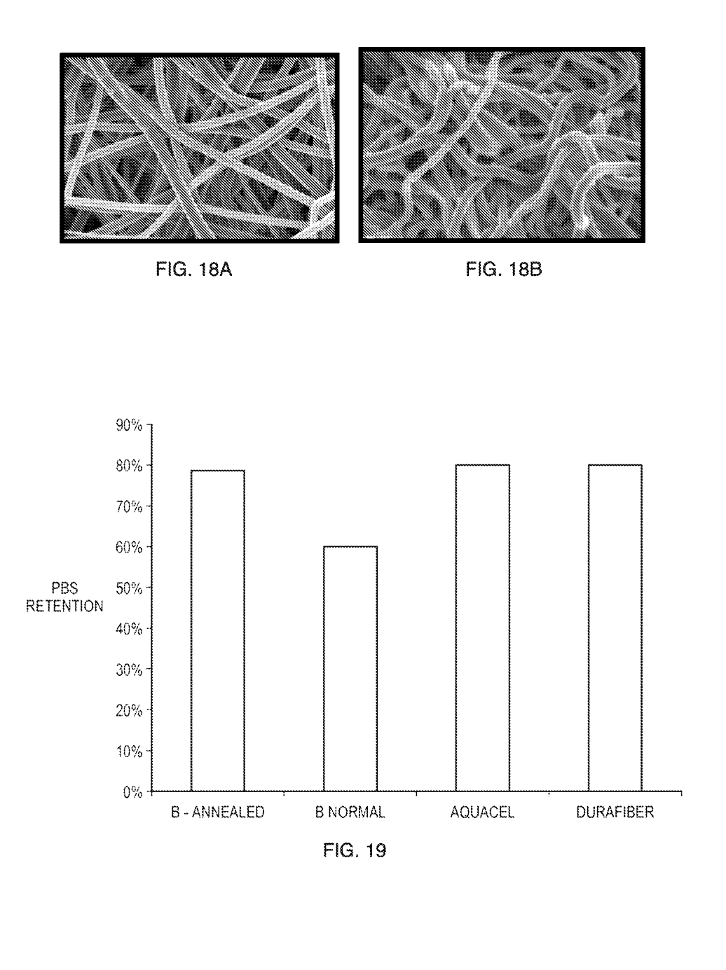

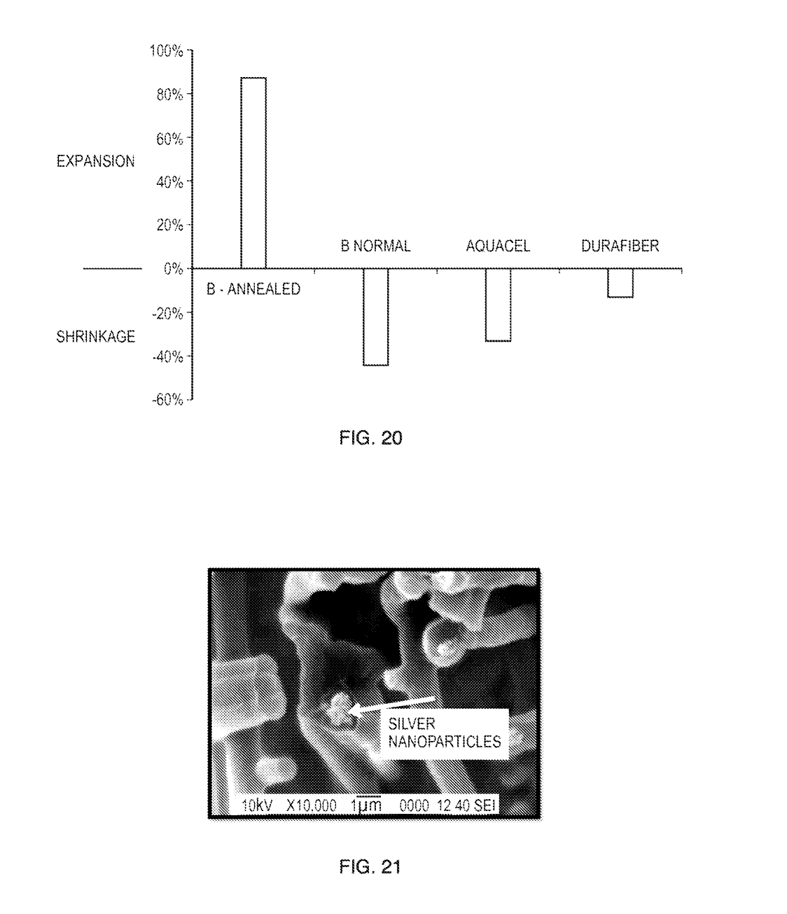

In various embodiments, meshes in accordance with the present disclosure are annealed at elevated temperature to improve the properties of the same. For example, HLPU/HBPU sheath/core fiber meshes as formed herein have been found to become less porous upon annealing. In this regard, FIGS. 18A and 18B are photomicrographs of a mesh formed from HLPU/HBPU sheath/core fibers as described herein, before and after annealing, respectively. Along with the reduction in mesh porosity, the annealing step is accompanied by a reduction in mesh volume (and thus mesh area). Unexpectedly, such an annealing step has been found to improve water retention and to result in mesh expansion (rather than mesh shrinkage). In this regard, FIG. 19 shows PBS retention values for non-annealed (B Normal) and annealed (B Annealed) HLPU/HBPU sheath/core fiber meshes in accordance with the present disclosure, as well as retention values for Aquacel® and Durafiber® wound dressings. As seen from FIG. 19, an annealed mesh material has been developed which provides PBS retention equivalent to that of Aquacel® and Durafiber® dressings. In this regard, FIG. 20 shows shrinkage or expansion values for non-annealed (B Normal) and annealed (B Annealed) HLPU/HBPU sheath/core fiber meshes in accordance with the present disclosure, as well as for Aquacel® and Durafiber® wound dressings. Thus, as seen from the foregoing, the present disclosure provides the ability to tailor mesh absorption, retention and shrinkage/expansion to the application at hand.

In addition, as noted elsewhere, the small fiber sizes obtained also improves softness, conformability and leads to very high surface areas. High surface area improves absorptive capabilities, hydration kinetics and drug release capabilities, among other properties. Moreover, the fibrous form factor allows for formation/collection into novel form factors such as yarns, ropes, tubes, meshes, etc.

### EXAMPLE 5

### Fibers with a Polyurethane Core Containing Silver Particles and a Hydrophilic Polyurethane Sheath

In this example, needle core-sheath electrospinning was employed, in which a hydrophilic aliphatic polyether-based thermoplastic polyurethane (HLPU) was used as the sheath material, while a mechanically stronger more hydrophobic aliphatic polyether-based thermoplastic polyurethane material (HBPU) was used as the core material. The electrospinning solutions were as follows: 4 wt % HLPU in TFE and 6 wt % HBPU in HFIP containing 30% silver nanoparticles with respect to the polymer. The resulting fibers exhibited a core-sheath geometry in which silver was encapsulated and are shown in FIG. 21. Silver is well-known for its antibacterial properties and such a mesh could be used for sustained release of silver for wound dressing applications. In addition to silver nanoparticles, other embodiments including incorporation of other particles and/or excipients into the core material to achieve different performance metrics. For example, cross-linked celluloses or other hydrophilic polymers can be incorporated into the core to further aid in the hydration properties of the resulting fiber.

### EXAMPLE 6

### Fibers with a Polyurethane Core and a Hydrophilic Polyurethane Sheath Containing Different Excipient Materials in the Sheath or Core

In this example, slit-surface core-sheath electrospinning was employed, in which an excipient material was added in the core of a core-sheath fiber to modulate its hydration properties. Similar to the above, a hydrophilic aliphatic polyether-based thermoplastic polyurethane (HLPU) was used as the sheath material, while a mechanically stronger more hydrophobic aliphatic polyether-based thermoplastic polyurethane material (HBPU) was used as the core material. The excipient material used to modulate hydration properties of the core was a cellulose gum, sodium carboxymethylcellulose (NaCMC). The electrospinning solutions were as follows: 4 wt % HLPU in TFE as the sheath solution and 6 wt % HBPU in HFIP containing either 50, 60, 70, or 80 wt % NaCMC as the core solution. The same electrospinning conditions were employed for each material system and as a result, four different formulations, each containing different amounts of NaCMC, were collected (Table 4). The inclusion of NaCMC had a small impact on increasing the absorption capacity of the fibers while playing a larger role in the retention properties. In the fibers of Example 4, the highest retention achieved was ˜60%, whereas in these embodiments, retention of 70% and greater were achieved. Moreover, the data indicates that the retention could be controlled by either increasing the amount of NaCMC content in the fiber or by increasing the basis weight of the material. When the NaCMC content increased from 24 to 32% (Formulation E vs. Formulation F), a 10% increase in retention was observed. Additionally, when the basis weight of the same material composition increased from 84 to 130 GSM (Formulation H vs. Formulation I), a 10% increase in retention was also achieved. Thus, the hydration properties can be controlled via a number of different mechanisms (e.g. material composition and physical form factor).

TABLE 4

| | Formulation | Formulation | Formulation | Formulation | Formulation |
|---|---|---|---|---|---|
| | E | F | G | H | I |
| HLPU:HBPU | 67:33 | 69:311 | 70:30 | 72:28 | 72:28 |
| Ratio | | | | | |
| NaCMC | 24% | 32% | 41% | 53% | 53% |
| content (%) | | | | | |
| Basis weight | 73 ± 2 | 92 ± 3 | 79 ± 1 | 84 ± 1 | 132 ± 6 |
| (GSM) | | | | | |
| PBS absorption | 1356 ± 20 | 1445 ± 9 | 1475 ± 64 | 1500 ± 4 | 1550 ± 50 |
| (%) | | | | | |
| PBS retention | 70 ± 3 | 81 ± 2 | 79 ± 1 | 82 ± 2 | 90 ± 3 |
| (%) | | | | | |

Alternate configurations of this concept were developed in which NaCMC and cross-linked sodium carboxymethylcellulose (croscarmellose sodium) was incorporated in the fiber. Moreover, the excipient material was incorporated in either the core or the sheath. Similar to the above, a hydrophilic aliphatic polyether-based thermoplastic polyurethane (HLPU) was used as the sheath material, while a mechanically stronger more hydrophobic aliphatic polyether-based thermoplastic polyurethane material (HBPU) was used as the core material. The electrospinning solutions were as follows: 4 wt % HLPU in TFE as the sheath solution and 6 wt % HBPU in HFIP. NaCMC or croscarmellose sodium was added to either the sheath or core solution and subsequently electrospun into fibers. Table 5 shows the different formulations that were fabricated and tested. The data indicates that a wide range of hydration and mechanical properties can be achieved by addition of different materials in either the core or the sheath.

TABLE 5

| | | | | Formulation L |
|---|---|---|---|---|
| | Formulation I | Formulation J | Formulation K | Croscarmellose |
| | NaCMC in the | Croscarmellose | NaCMC in the | sodium in the |
| | core | sodium in the core | sheath | sheath |
| Amount of | 53% | 20% | 38% | 48% |
| cellulose content | | | | |
| in fiber | | | | |
| PBS absorption | 1550 | 1320 | 1660 | 1320 |
| (%) | | | | |
| Retention (%) | 89% | 54% ± 10% | 90% ± 3 | 68 ± 4% |
| Shrinkage (%) | 26 ± 4 | −12 ± 7 | 51 ± 2 | 49 ± 3% |
| Wet tensile | 0.087 ± 0.008 | 0.355 ± 0.055 | 0.044 ± .004 | 0.031 ± 0.003 |
| strength (MPa) | | | | |

Although various aspects and embodiments are specifically described herein, it will be appreciated that modifications and variations of the present invention are covered by the above teachings and are within the purview of the appended claims without departing from the spirit and intended scope of the invention.

1. A multicomponent fiber comprising (a) a polymeric core that comprises a core-forming polymer and (b) a polymeric sheath that comprises a hydrophilic polymer, wherein said core-forming fiber is more hydrophobic than said hydrophilic polymer and wherein said polymeric core, said polymeric sheath, or both, further comprises a hydrophilic excipient material.

2. The multicomponent fiber of claim 1, wherein the hydrophilic excipient material is a cross-linked or non-cross-linked hydrophilic polymer that is different from the hydrophilic polymer and the core-forming polymer.

3. The multicomponent fiber of claim 2, wherein the cross-linked or non-cross-linked hydrophilic polymer is a cross-linked or non-cross-linked hydrophilic natural polymer.

4. The multicomponent fiber of claim 3, wherein the cross-linked or non-cross-linked hydrophilic natural polymer is a cross-linked or non-cross-linked polysaccharide.

5. The multicomponent fiber of claim 4, wherein the cross-linked or non-cross-linked polysaccharide is selected from cross-linked and non-cross-linked cellulose and cross-linked and non-cross-linked cellulose derivatives.

6. The multicomponent fiber of claim 4, wherein the cross-linked or non-cross-linked polysaccharide is selected from carboxymethyl cellulose, salts of carboxymethyl cellulose, cross-linked carboxymethyl cellulose and salts of cross-linked carboxymethyl cellulose.

7. The multicomponent fiber of claim 1, wherein said multicomponent fiber is formed by a core-sheath electrospinning process.

8. The multicomponent fiber of claim 1, wherein the multicomponent fiber ranges from 0.1 to 20 microns in diameter.

9. The multicomponent fiber of claim 1, wherein the ratio of sheath volume to core volume in the multicomponent fiber ranges from 100:1 to 1:1.

10. The multicomponent fiber of claim 1, wherein the hydrophilic polymer is covalently crosslinked.

11. The multicomponent fiber of claim 1, wherein the hydrophilic polymer is a hydrophilic polyurethane.

12. The multicomponent fiber of claim 11, wherein the hydrophilic polyurethane is an aliphatic, polyether-based polyurethane.

13. The multicomponent fiber of claim 1, wherein the core-forming polymer is a thermoplastic polymer.

14. The multicomponent fiber of claim 1, wherein the core-forming polymer is an aliphatic polyether-based thermoplastic polyurethane.

15. The multicomponent fiber of claim 1, wherein the core-forming polymer is a crosslinked polysiloxane.

16. A nonwoven mesh formed by the multicomponent fiber of claim 1.

17. The mesh of claim 16, wherein the mesh ranges from 10 to 5000 microns in thickness and the multicomponent fiber ranges from 0.1 to 20 microns in diameter.

18. The mesh of claim 16, wherein the mesh has a modulus wet tensile strength of at least 0.005 MPa.

19. The mesh of claim 16, wherein upon immersion in aqueous medium at 25° C. for one hour, the mesh has an absorbency ranging from 500% to 2500%.

20. The mesh of claim 16, wherein the porosity of the mesh is less than 99%.

21. A medical article comprising the mesh of claim 16.

22. A method for forming the multicomponent fiber of claim 1, comprising electrospinning said multicomponent fiber from a first solution comprising said hydrophilic polymer and a second solution comprising said core-forming polymer, wherein the first solution, the second solution, or both, comprises said hydrophilic excipient material.
